(19) United States
(12) Patent Application Publication
(10) Pub. No.: US 2004/0044288 A1
(43) Pub. Date: Mar. 4, 2004

(54) Apparatus and method for non-invasive monitoring of cardiac output

(76) Inventors: Miguel Gorenberg, Haifa (IL); Hector Rotstein, Haifa (IL); Michael Naroditzky, Carmiel (IL); Alon Marmor, Carmiel (IL); Ehud Dafni, Caesaria (IL)

Correspondence Address: Reed Smith LLP, 29th Floor, 599 Lexington Avenue, New York, NY, 10022, US

(21) Appl. No.: 10/234,429

(22) Filed: Sep. 3, 2002

(51) Int. Cl. A61B 5/02

(52) U.S. Cl. 600/481; 600/499

(57) ABSTRACT

A non-invasive apparatus for measuring cardiac mechanical performance of a patient, the apparatus comprising a pressure applying element mountable on a limb of the patient for applying pressure high enough to make a segment of an artery within the limb achieve a collapsed state and empty it from blood at least momentarily; at least one of a plurality of sensors coupled to said pressure applying element, sensing mechanical changes corresponding to volumetric changes in the artery as the artery progressively recuperates from its collapsed state; processing unit communicating with said at least one of a plurality of sensors for receiving output corresponding to the mechanical changes from said at least one of a plurality of sensors and computing factors correlated with blood flow and calculate parameters indicating heart performance.

### FIELD OF THE INVENTION

The present invention is related to non-invasive monitoring of heart mechanical performance. More particularly, the present invention is related to a noninvasive apparatus and method for measuring the mechanical performance of the heart using periodic or continuous monitoring and recording the flow of blood by peripherally mounted arterial sensors.

### BACKGROUND OF THE INVENTION

Heart muscle ischemia due to coronary artery diseases is one of the leading causes of death in the world; in the United States alone, it affects more than 13 million people. Myocardial ischemia can be defined as a decrease in the supply of blood to the heart, and more precisely as an imbalance between the supply and demand of myocardial oxygen. In most clinical situations, the reason for this imbalance is inadequate perfusion of the myocardium due to obstructions or stenosis of the coronary arteries. The ischemia can last a few seconds or persist for minutes or even hours, causing transient or permanent damage to the heart muscle. The population that suffers ischemic heart diseases is at high risk of recurrent myocardial infarction. Each year, an estimated amount of 1 million Americans will have a new or recurrent coronary attack while more than 40% of the people experiencing coronary attack are expected to die resulting from it.

In order to monitor ischemic incidents and especially recurring ones, population at risk may connect to a cardiac center through a telephone line. Today, ambulatory monitoring of these patients or elderly population is performed using trans-telephonic electrocardiography (TTE). Patients experiencing suspected symptoms can correlate these symptoms with their ECG's at the time they are experiencing the incident and than transmit their ECG through the telephone line to the cardiac center for assessment.

There are several disadvantages in using TTE:

1. TTE requires the patient to be symptomatic when experiencing a cardiac event. However, 40-70% of transient ischemic episodes are silent, not associated with anginal chest pain or any other symptoms. A patient experiencing a silent episode will most probably not be aware of his situation and consequently will not use TTE.

2. TTE requires the patient to connect electrodes to his body, activate a recorder, and at the same time to phone the cardiac center and trans-telephonic transmit the ECG. This is a complicated and an error-prone procedure, especially when performed by a patient suffering from these symptoms.

3. the ECG test was shown in studies to have low sensitivity for diagnosis of ischenia (about 60%). It has been shown that even patients with clear symptomatology may have a normal ECG.

Experimental and clinical studies in the cardiologic literature and other references indicate that changes in the cardiac mechanical performance occur relatively early when an incidence of ischemia takes place, and indexes reflecting the mechanical performance of the heart are more sensitive than Electrocardiographs (ECG) changes or subjective symptoms for detecting myocardial ischemia. See: Kayden et al., “Validation of Continuous Radionucleide Left Ventricular Functioning Monitoring in Detecting Silent Myocardial Ischemia during Balloon Angioplasty of the Left Anterior Descending Coronary Artery”, Am. J. Cardiol. 67, 1339-1343 (1991), In this work the authors used balloon inflation in the course of transluminal coronary angioplasty as a human model of transient myocardial ischemia due to acute reduction of coronary blood flow, showing that 17/18 inflation were associated with a significant decrease in Left Ventricular Ejection Fraction, in contrast, there was chest pain in only 10 inflation's and ECG changes in 7.

Decrease in blood flow in a peripheral artery during transient myocardial ischemia has been demonstrated in the arm of patients during balloon inflation in the course of transluminal coronary angioplasty using a non-invasive venous occlusion plethysmography, See: Indolfi et. al. “Limb vasoconstriction after successful angioplasty of the left anterior descending coronary artery”, Circulation 92, 2109-2112 (1995). The same phenomena was demonstrated in dogs by measuring the flow in the femoral artery. In that case, the flow was measured by placing an electromagnetic probe around the left femoral artery after selective embolization of the left coronary artery with mercury. See: Falicov et.al. , “The response of the renal and femoral vascular beds to coronary embolization in the dog”, Cardiovascular Research 9, 151-160 (1975).

As explained above, it is very desirable to provide a device that could monitor cardiac mechanical performance, especially among the population at risk. Monitoring should be performed either periodically or continuously, independently of clinical symptomatology. As a consequence of the two observations in the previous paragraph, such monitoring can be performed by measuring changes in blood flow in the arm non-invasively and therefore providing an early detection of the cardiac pump impairment induced by ischemia. Noninvasive measurements of blood flow are usually performed using Doppler technique in which ultrasonic sound waves are transmitted through the skin roughly parallel to the blood flow direction, and variations in the ultrasonic frequency are sensed to determine the blood flow velocity. The Doppler technique has several inherent limitations: it measures blood flow velocity in velocity units rather than the desired volumetric blood flow quantity which is in volume per time units. In addition, signal-to-noise considerations limit the accuracy of the measurement. The measurement is dependent on the location of the sensor with respect to the blood vessel and in order to establish an accurate measurement, the patient has to remain still throughout the measurement. As opposed to Doppler sensors, electromagnetic blood meters have the advantage of insensitivity to the patient's movements and to the contact angle. Doppler ultrasound measurements are time consuming and must be performed by trained healthcare professionals. Hence, this technique does not provide a solution for continuous or ambulatory monitoring.

Alternative solutions for monitoring of the cardiac performance are based on various electromagnetic sensors. Examples of electromagnetic sensors were disclosed in U.S. Pat. No. 4,412,545 by Okino et al. “Electromagnetic Blood Flowmeter” and in PCT/IL01/00583 (Gorenberg et al.), titled APPARATUS AND METHOD FOR NON-INVASIVE MONITORING OF HEART PERFORMANCE, published as WO/02/00094.

Additional sensors for non-invasive measurements of hemodynamic parameters have also been proposed. The patents U.S. Pat. No. 5,095,912 (Tomita), U.S. Pat. No. 5,301,675 (Tomita), U.S. Pat. No. 5,316,005 (Tomita), U.S. Pat. No. 5,388,585 (Tomita), U.S. Pat. No. 5,406,954 (Tomita), U.S. Pat. No. 5,423,324 (Tomita), U.S. Pat. No. 5,651,369 (Tomita) and U.S. Pat. No. 6,231,523 (Tomita), disclose pressure measurement devices coupled with cuffs over the upper arm, resembling in some ways certain embodiments of the present invention. However, the inventions by Tomita are different than the present invention. Tomita measures the time delay between the pressure pulses at two points along the arm. The results are a momentary measurement of pressure pulse propagation at an ill defined blood pressure and are hard to correlate to any hemodynamic parameter known in the art. The present invention does not have that difficulty, since it measures other parameters and it is based upon different principles as will be explained hereinafter. The present invention is different both is essence and in details from Tomita also in mechanical configuration, the specific blood pressure at which the measurement is performed and the algorithms for data processing.

The patents U.S. Pat. No. 6,319,205 (Goor) and U.S. Pat. No. 6,322,515 (Goor) disclose an apparatus and method for monitoring physiological changes by performing a continuous monitoring of the arterial tone at the digit of the subject. Some of the embodiments in Goor involve mounting a cuff around the digit, application of pressure and monitoring the tone at the extreme end of the digit. However, also this invention is different in essence and in details from the present invention as explained hereinafter.

The patent U.S. Pat. No. 5,503,156 (Millar) disclose a noninvasive pulse transducer for simultaneously measuring pulse pressure and velocity. The sensor disclosed in Millar's invention may have application in hemodynamic measurements but it is not specifically related to the present invention.

As explained herein below, the object of the present invention can also be used to determine relative cardiac output under stress. The techniques currently in use for detecting myocardial ischemia elaborated during exercise tests are summarized next:

1) ECG (Electrocardiography): The ECG depicts abnormal electrical activities which may arise in ischemic myocardial regions. The sensitivity and specificity of the ECG in detecting myocardial ischemia is directly related to the extensiveness of the arteriosclerotic disease. Hence, a high risk localized disease (i.e. limited to one or two arterial branches) may be overlooked and the overall predictive value of ECG in stress test is only approximately 60%.

2) Stress echocardiography with Dobutamine infusion: This technique is based on performing two-dimensional ultrasonic imaging of the walls of the heart while infusing controlled doses of Dobutamine. During the stress testing the myocardial segments related to arteriosclerotic coronary arteries may become ischemic. Consequently, wall motion disturbances, such as hypokinesia and/or a decrease in wall thickening, may be depicted by the echocardiograph as well decrease in aortic blood flow and ejection fraction. Continuing improvements in this technique have increased the predictive diagnostic value of stress echo to approximately 75%-80%, which is nearly as high as nuclear imaging technologies (see inhere below). The test can be performed in the doctor's office but since it is labor intensive and professionally demanding it is not appropriate for ambulatory monitoring.

3) Nuclear imaging technologies. Radioactive isotopes are injected intravenously at peak physical effort or after the induction of pharmacological effort by Dobutamine infusion. A second intravenous dose of the same isotope is applied after the first dose is washed out and the patient is at rest. That procedure enables the physician to distinguish between filling defects due to infarcted regions versus transient filling defects in demand-related ischemic segments.

A specific example of a nuclear technique is Tc99-Sestamibi-SPECT (Single Photon Emission Computed Tomography). The injected Tc99-Sestamibi is a radioactive tracer which is “absorbed” by the viable myocardial cells. In infarcted or inadequately perfused ischemic regions under stress, Tc99-Sestamibi uptake by the myocardium is stopped, and appears as filling defects. In a second scan performed a few hours after the can under stress, uptake of the radioactive tracer can be seen in previously ischemic regions.

At the present time, the nuclear methods are the best available non-invasive procedures in clinical routines for ischemia detection for use after a positive result was obtained with ECG, or based on the physician's assessment of the patient. Reliability is in the range of 82-85%.

Patients deemed to have a significant degree of demand related myocardial ischemia on the basis of the diagnostic tests described herein above are usually further referred for cardiac catheterization and coronary angiography, which is the most invasive, but also the most definitive diagnostic test available.

As explained herein below, the object of the present invention can further be used to determine sleep apnea. The following explanation of apnea appeared in Goor (U.S. Pat. No. 6,322,515). Sleep apnea syndrome is one of the most common and serious sleep disorders. It is characterized by repetitive episodes of upper airway collapse during sleep resulting in interruption of airflow despite persistent respiratory effort. Obstructive apneas are typically associated with progressively increasing asphyxia until termination by a brief arousal from sleep and restoration of upper airway patency. Population studies have estimated that 2-4% of the adult population suffer from sleep apnea syndrome. The syndrome has been identified as an important risk factor to systemic hypertension, myocardial infarction, stroke, and sudden death. To diagnose sleep apnea syndrome, usually simultaneous recordings are made on a multi-channel recorder consisting of an electroencephalogram (EEG), electro-oculogram (EOG), submental electromyogram (EMG), oro-nasal airflow (by thermistors or thermocouples) and thoraco-abdominal movements (by respiratory belt), snoring intensity (by dB meter), pulse oximetry and leg movements. Each record is scored visually for all apneic events. The recordings are cumbersome and may interfere with the sleep of the patients. In view of the difficulties with existing sleep evaluation techniques, there are many cases in which only partial monitoring is conducted, consisting only of respiratory effort and oximetry. Partial recordings are done particularly for screening purposes. Their purpose is to identify persons with large numbers of apneic events.

Accordingly, there is a need for a simpler method for sleep staging and sleep apnea syndrome detection, which would allow the patient to sleep comfortably during the evaluation.

The present invention deals with the measurements of hemodynamic parameters known in the art. The hemodynamic parameters related to the present invention are defined next:

The stroke work (SW) is the external work performed by the left ventricle of the heart in one heart cycle and is calculated as the area of the pressure/volume loop. The pressure/volume loop is obtained by plotting the variations of the volume as a function of the pressure over one heart cycle. It can be approximated as

SW≈SV×MAP

Here MAP denotes the mean artery pressure and SV the stroke volume.

It follows that the stroke work integrates the two determinants of perfusion: flow and pressure.

Since the measurements of the apparatus disclosed by the present invention are preferably performed on a peripheral artery, the peripheral stroke volume (PSV) is to be estimated. Assuming that the diameter of the peripheral artery used for the measurement does not change significantly between heart cycles, the PSV is calculated by multiplying the integrated velocity curve by the artery area.

The cardiac output (CO) is the amount of blood pumped by the left ventricle each minute. The peripheral CO, namely the fraction of CO reaching the peripheral section, can be calculated by multiplying the PSV by the heart rate (HR).

The peripheral stroke work is calculated by multiplying the PSV by the peripheral MAP.

The Peripheral Vascular resistance (PVR) is calculated by dividing the MAP by the peripheral CO.

The Velocity Time Integral (VTI) is the integral of the velocity-time curve of the blood at the output of the left ventricle of the heart, over one heart cycle. Note that the SV can be estimated from the product of the VTI times the mean aortic cross section, hence the VTI measures an important parameter of the mechanical functioning of the heart. The peripheral VTI is the VTI measured on a peripheral artery. As known in the art, there is a strong correlation between VTI and PVTI on patient's limbs.

### BRIEF DESCRIPTION OF THE INVENTION

It is an object of the present invention to provide a new and unique noninvasive device and method for monitoring, periodically or continuously, the heart mechanical performance. The main object is to compute blood flow through the measurement of the velocity time integral (VTI), but other indexes that reflects the cardiac performance can be estimated as well, including peripheral stroke volume (PSV), peripheral cardiac output (CO) peripheral stroke work (PSW) and Peripheral Vascular Resistance (PVR).

It is another object of the present invention to provide a new and unique device and method for monitoring the mechanical performance of the heart while the device is preferably mounted on the upper arm, the lower arm or the wrist, so that comfortable measurements conditions are met. The device may be mounted on another peripheral organ or area that meets the requirements of which blood flow may be measured without interference.

It is an additional object of the present invention to provide a new device that alerts patents to seek for immediate medical assistance when their heart performance is deteriorating.

It is yet another object of the present invention to provide a new device that facilitates true diagnosis in cases of ischemia so that false positive and false negatives ECG interpretation is avoided.

An additional object of the present invention is to provide a new device and method that facilitates evaluation of ischemia severity.

Yet, it is an additional object of the present invention to provide a new and unique device and method for recording and storing synchronized ECG signals with parameters that are correlated to the mechanical cardiac performance for relatively long periods of time (24-48 hours or even more) so as to provide an improved Holter system.

It is yet another object of the present invention to provide a new device to facilitate the diagnosis of obstructive sleep apnea syndrome by monitoring changes in peripheral vascular resistance (PVR).

There is thus provided, in accordance with a preferred embodiment of the present invention, a non-invasive apparatus for measuring cardiac mechanical performance of a patient, the apparatus comprising:

a pressure applying element mountable on a limb of the patient for applying pressure high enough to make a segment of an artery within the limb achieve a collapsed state and empty it from blood at least momentarily;

at least one of a plurality of sensors coupled to said pressure applying element, sensing mechanical changes corresponding to volumetric changes in the artery as the artery progressively recuperates from its collapsed state;

a processing unit communicating with said at least one of a plurality of sensors for receiving output corresponding to the mechanical changes from said at least one of a plurality of sensors and computing factors correlated with blood flow and calculate parameters indicating heart performance.

Furthermore, in accordance with a preferred embodiment of the present invention, wherein the pressure applying element is an inflatable cuff.

Furthermore, in accordance with a preferred embodiment of the present invention, the pressure applying element is an inflatable cuff, divided into a plurality of inflatable segments.

Furthermore, in accordance with a preferred embodiment of the present invention, the inflatable cuff is divided into at least two inflatable segments, and wherein said at least one of a plurality of sensors comprise at least two sensor transducers for detecting pressure changes within the segment, each transducer corresponding to a different segment.

Furthermore, in accordance with a preferred embodiment of the present invention, the pressure applying element is operated by a pneumatic system comprising a pump for increasing the pressure within the cuff, and valves for releasing the pressure from the cuff.

Furthermore, in accordance with a preferred embodiment of the present invention, the pressure applying element is driven by an electrical motor.

Furthermore, in accordance with a preferred embodiment of the present invention, the pressure applying element is coupled to a bracelet having a diameter which is automatically adjustable.

Furthermore, in accordance with a preferred embodiment of the present invention, the bracelet consists of a strap and wherein bracelet's diameter may be increased or decreased by turning a screw operated by a motor to which the strap is attached.

Furthermore, in accordance with a preferred embodiment of the present invention, the pressure applying element is hydraulically operated.

Furthermore, in accordance with a preferred embodiment of the present invention, the pressure applying element comprises said at least one of the plurality of cushions held against the limb by a rigid bridge.

Furthermore, in accordance with a preferred embodiment of the present invention, the cushions are inflatable.

Furthermore, in accordance with a preferred embodiment of the present invention, said at least one of the plurality of cushions consist of two such cushions, filled with filled with ferromagnetic fluid that transforms from liquid to solid by application of magnetic flux, and electromagnetic coil provided adjacent each cushion, for inducing magnetic flux.

Furthermore, in accordance with a preferred embodiment of the present invention, the pressure applying element comprises at least one of a plurality of cushions held against the limb by a rigid bridge, and wherein said at least one of a plurality of sensors comprises deformation sensors, sensing deformation changes of said at least one of the plurality of cushions.

Furthermore, in accordance with a preferred embodiment of the present invention, said at least one of the plurality of cushions is inflatable.

Furthermore, in accordance with a preferred embodiment of the present invention, said at least one of the plurality of cushions is filled with hydraulic fluid.

Furthermore, in accordance with a preferred embodiment of the present invention, the deformation sensors comprise an array of capacitors wherein the mechanical changes are determined by measuring changes in the capacitance of the capacitors, due to deformation changes.

Furthermore, in accordance with a preferred embodiment of the present invention, the pressure applying element comprises at least one cushion held against the limb by at least one of a plurality of pivotal rigid bridges, provided with gyroscopic sensor to sense rotational velocity of said at least one of a plurality of pivotal rigid bridges.

Furthermore, in accordance with a preferred embodiment of the present invention, said at least one of a plurality of pivotal rigid bridges comprise two pivotal bridges.

Furthermore, in accordance with a preferred embodiment of the present invention, the two pivotal bridges are coupled to a third pivotal bridge.

Furthermore, in accordance with a preferred embodiment of the present invention, said at least one of a plurality of sensors include an array of piezoelectric transducers.

Furthermore, in accordance with a preferred embodiment of the present invention, the apparatus further comprises output means.

Furthermore, in accordance with a preferred embodiment of the present invention, the apparatus further comprises memory unit.

Furthermore, in accordance with a preferred embodiment of the present invention, the apparatus further comprises means to communicate with a computer, network or a telephone system.

Furthermore, in accordance with a preferred embodiment of the present invention, the pressure applying element is capable of applying pressure sufficient to cause a collapse of the artery just momentarily during a diastolic phase of the patient.

Furthermore, in accordance with a preferred embodiment of the present invention, the processing unit includes algorithm comprising the following steps:

a. calculating instantaneous pressure changes within the pressure inducing member as a function of time;

b. dividing the instantaneous pressure changes into segments corresponding to pulse rate periods of the patient and normalizing the pressure changes of each time segment;

c. finding the highest pressure at which where there exists no separation between the falling edge and leading edge of two consecutive segments of the normalized instantaneous pressure changes and analyzing at least one segment located within 5 pulse rates from the two consecutive segments.

Furthermore, in accordance with a preferred embodiment of the present invention, the algorithm included in the processing means further comprises, in the presence of noise, measuring and tabulating values of time elapsed between two pulses at a predetermined threshold and extrapolating the highest pressure at which there exists no separation between the falling edge and leading edge of two consecutive segments of the normalized instantaneous pressure changes.

Furthermore, in accordance with a preferred embodiment of the present invention, the highest pressure at which there exists no separation between the falling edge and leading edge of two consecutive segments of the normalized instantaneous pressure changes is found by first increasing the applied pressure above the desired pressure and than acquiring pressure data while gradually reducing the applied pressure.

Furthermore, in accordance with a preferred embodiment of the present invention, the highest pressure at which there exists no separation between the falling edge and leading edge of two consecutive segments of the normalized instantaneous pressure changes is found by gradually increasing the applied pressure while acquiring pressure data.

Furthermore, in accordance with a preferred embodiment of the present invention, a control system is used to maintain the applied pressure over a period of time substantially at the highest pressure at which where there exists no separation between the falling edge and leading edge of two consecutive segments of the normalized instantaneous pressure and factors correlated with blood flow are measured continuously.

Furthermore, in accordance with a preferred embodiment of the present invention, the measurement data is used to calculate the peripheral velocity time integral PVTI.

Furthermore, in accordance with a preferred embodiment of the present invention, the PVTI is calculated by a fit of a theoretical curve to the combined data of plurality of sensors, each detecting pressure changes within corresponding segment of the inflatable cuff.

Furthermore, in accordance with a preferred embodiment of the present invention, the PVTI is calculated from the time difference between data of plurality of sensors, each detecting pressure changes within corresponding segment of the inflatable cuff.

Furthermore, in accordance with a preferred embodiment of the present invention, the PVTI is calculated by a fit of a theoretical curve to data indicating sensor segment triggering time versus said segment position.

Furthermore, in accordance with a preferred embodiment of the present invention, PVTI data is used to calculate further factors correlated with blood flow.

Furthermore, in accordance with a preferred embodiment of the present invention, there is provided a method for non-invasive measuring of changes in cardiac mechanical performance of a patient, the method comprising:

providing a pressure applying element mountable on a limb of the patient for applying pressure enough to make a longitudinal segment of an artery within the limb achieve a collapsed state and empty it from blood at least momentarily;

providing sensor coupled to the pressure applying element, sensing mechanical changes corresponding to volumetric changes in the artery as the artery progressively recuperates from its collapsed state;

providing processing unit communicating with the sensor for receiving output corresponding to the mechanical changes from the sensor and computing factors correlated with blood flow and calculate parameters indicating heart performance;

applying pressure on a portion a limb of a patient through which artery passes enough to collapse the artery preventing at least momentarily the flow of blood through the collapsed artery;

sensing mechanical changes corresponding to volumetric changes in the artery as the artery progressively recuperates from its collapsed state;

computing factors correlated with blood flow and calculating parameters indicating heart performance.

Furthermore, in accordance with a preferred embodiment of the present invention, the pressure applied on the portion of the limb of the patient is initially larger than needed to collapse the artery, and wherein it is gradually reduced, sensing the mechanical changes correlating to the volumetric changes while the pressure is reduced.

Furthermore, in accordance with a preferred embodiment of the present invention, the method further comprises determining a best pulse period for considering a measurement, comprising the steps of:

a. calculating instantaneous pressure changes within the cuff as a function of time;

b. dividing the instantaneous pressure changes into segments corresponding to pulse rate periods of the patient and normalizing the pressure changes of each time segment;

c. finding two consecutive segments of the normalized instantaneous pressure changes where there exists no separation and analyzing at least one segment located within 5 pulse rates from the two consecutive segments.

Furthermore, in accordance with a preferred embodiment of the present invention, the method further comprises measuring blood pressure of the patient.

Furthermore, in accordance with a preferred embodiment of the present invention, the method further comprises measuring heart pulse rate of the patient.

Furthermore, in accordance with a preferred embodiment of the present invention, the method steps are carried out continuously over a period of time, in order to diagnose heart performance disorders.

Furthermore, in accordance with a preferred embodiment of the present invention, the method further comprises transmitting data to an external apparatus.

Finally, in accordance with a preferred embodiment of the present invention, the method is incorporated with Holter procedure, in order to detect artifacts and enhance reliability.

Further features of the present invention are explained herein below.

### BRIEF DESCRIPTION OF THE DRAWINGS

FIG. 1 illustrates propagating blood flow in a peripheral blood vessel.

FIG. 2 shows a graph illustrating the variation of an artery cross-section as a function of the pressure on the artery's walls.

FIG. 3a shows three stages in the progress of blood through a collapsed artery.

FIG. 3b is a graph showing the changes in volume within the collapsed artery as blood progresses through the collapsed artery.

FIG. 4 illustrates the variation in the internal pressure of the cuff, as the static pressure of the cuff is reduced. The peaks are caused by the artery when recovering from the collapsed state.

FIG. 5 illustrate a noninvasive device for monitoring heart mechanical performance in accordance with a preferred embodiment of the present invention, worn on the upper arm.

FIG. 6 illustrate a noninvasive device for monitoring heart mechanical performance in accordance with a preferred embodiment of the present invention, worn on the wrist.

FIG. 7 illustrates a schematic diagram of the pneumatic components of a monitoring device in accordance with a preferred embodiment of the present invention.

FIG. 8 illustrates a schematic diagram of the electronic components of a monitoring device in accordance with a preferred embodiment of the present invention.

FIGS. 9-13 illustrate cross-sectional views of the progress of blood through a collapsed artery, and the induced mechanical changes within the cuff (the pressure changes within the cuff are shown in a chart below each drawing).

FIG. 14 illustrates a graph of the variations in internal pressure within a segment of the cuff following the opening of the collapsed artery. The graph is obtained after subtracting the static pressure that reduces monotonically, hence the 0-baseline.

FIG. 15 illustrates a graph of the variations in internal pressure within a segment of the cuff, corresponding to the FIG. 14, with the minimum and maximum local values normalized between 0 and 1.

FIG. 16 illustrates the normalized pressure reading output of the two cuff segments. The middle point, corresponding to an abscissa of 0.5 is important since the measurement is linear there and the middle point of the second cuff segment should correspond to a value of 1.5 of the first segment.

FIG. 17 illustrates how the output of the second segment is lifted above the output of the first segment, to allow measurement of the progress of the recovery of the artery from collapsed state in a continuous manner.

FIG. 18 illustrates how the output of the first segment, and the output of the second segment after been lifted are interpolated using a continuous function.

FIG. 19 illustrates the location for positioning the monitoring device over a wrist, in accordance with a preferred embodiment of the present invention.

FIGS. 20a and 20b illustrate the pressure applied on an artery in the wrist and the progress of blood through the artery.

FIG. 21a illustrates a pressure applying structure with two cushions coupled to a bridge.

FIG. 21b shows another embodiment of the present invention, where the cushions are filled with fluid.

FIG. 21c shows the details of a proposed control unit for the embodiment shown in FIG. 21b.

FIGS. 22a and 22b illustrate a pressure applying structure with a single cushion with deformation sensors (22a) and typical electronic scheme (22b).

FIG. 22c shows details of a mechanism for applying external pressure in an embodiment based on the structure disclosed in FIG. 22a or other embodiments of the present invention, where the cushion is filled with hydraulic fluid material.

FIG. 22d shows typical capacitance-time curves obtained using the embodiment shown in FIG. 22a.

FIG. 22e illustrates a graph of pulse time versus capacitor position obtained using the embodiment shown in FIG. 22a.

FIG. 23 illustrates a double-cushion pressure applying structure with a gyroscopic sensor.

FIG. 24 illustrates a gyroscopic pressure applying structure with two wings connected by a bridge.

FIG. 25 illustrates a pressure applying structure incorporating two structures as shown in FIG. 24, coupled to a bridge.

FIG. 26 illustrates a pressure applying structure with two cushions filled with ferromagnetic fluid and with electromagnetic coil actuators, and gyroscopic sensor.

FIG. 27 illustrates a suggested diagram of the electronic scheme of the monitoring device whose pressure applying structure is shown in FIG. 26.

FIGS. 28-29 illustrate different stages in the progress of blood through an artery and the operation of the pressure applying structure of FIG. 26.

FIG. 30a shows a schematic structure of another preferred embodiment of the present invention with piezoelectric sensors.

FIG. 30b illustrates data acquisition circuitry for the embodiment of FIG. 30a.

FIG. 31a shows typical voltage-time curves obtained using the embodiment shown in FIG. 30a.

FIG. 31b illustrates a graph of pulse time versus piezoelectric sensor position obtained using the embodiment shown in FIG. 30a.

### DETAILED DESCRIPTION OF THE INVENTION AND DRAWINGS

The present invention provides a noninvasive device and method for peripheral monitoring of the mechanical performance of the heart muscle in a periodic manner, continuously or per user request. The noninvasive monitoring device is relatively small in dimensions, therefore portable and may be designed as a cuff or bracelet that may be worn on the upper arm, lower arm, or wrist of a patient, acquire information and store it or transmit it to a processing or display device.

The inventors of the present invention found and demonstrated a clear and distinct correlation between indexes of heart performance measured centrally and peripherally, therefore, the convenience of such a device is apparent and appealing. See: PCT/IL01/00583 (Gorenberg et al.), APPARATUS AND METHOD FOR NON-INVASIVE MONITORING OF HEART PERFORMANCE, published as WO/02/00094

The principle of the invention can be best understood by referring to FIG. 1. At time t₁ (upper part) an elementary blood volume dV (22) is at rest corresponding to the diastolic pressure when the blood flow at the artery 20 reduces to zero. The blood flow occurring between two diastole, t₁ and t₂ (lower part) in the Figure, produces a net displacement of dV by an amount of

$$l = {\int }_{{t}_{1}}^{{t}_{2}} v ( τ ) ⅆ τ .$$

Consequently, a sensor measuring l is actually measuring the peripheral velocity time integral at the given point, and hence an indication of the VTI and heart performance.

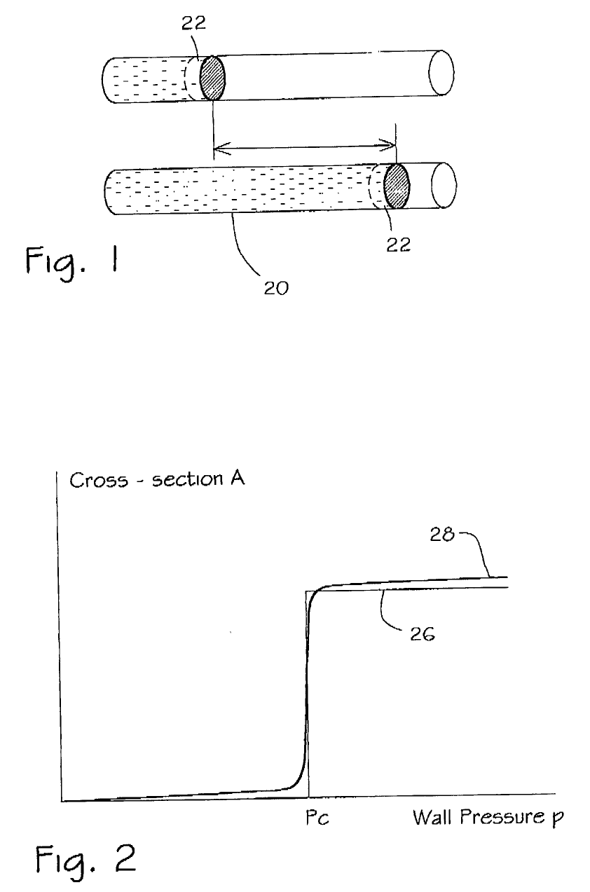

In order to measure l, the invention disclosed herein uses the collapsible nature of arteries to empty the vessel from blood in between diastole. A sufficiently large negative pressure on an artery wall makes it collapse, emptying the vessel from blood. At a critical pressure, p_{c}, the vessel recovers its original form hence allows the free circulation of blood. In FIG. 2, curve 28 schematically shows the variation of an artery cross section as a function of the pressure on the artery's walls. In some embodiments of the present invention it is assumed that the artery changes from fully closed to fully open at a given critical pressure, as shown in FIG. 2 curves 26. This situation is idealized, and consequently the measurements can be corrected in other embodiments using appropriate algorithm. However, the inventors found the simplified model to provide adequate results even without such corrections.

Now suppose that a sufficiently large external pressure is applied so that the pressure at the artery reaches p_{c} and therefore collapses. This external pressure can be achieved by means of an external device applying pressure on the organ where the artery is located, preferably the arm or wrist, but also in any other peripheral part of the body (preferably, but not limited to a limb). When the internal pressure peak driving the blood flow reaches the section where the external pressure is applied, it fully opens the artery, thus allowing the free flow of blood. If the external pressure is applied in a distributed manner along the artery, then the vessel will be opened by the blood flow as the elementary blood volume progresses.

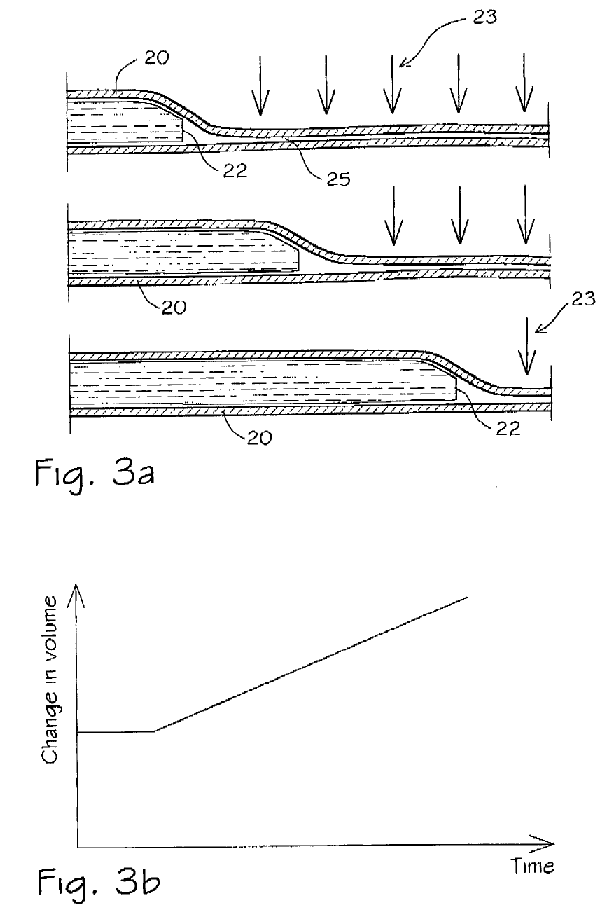

The principle is illustrated in FIG. 3a and FIG. 3b, FIG. 3a showing three stages in the progress of blood through the collapsed artery and FIG. 3b showing the changes in volume within the collapsed artery as blood progresses through the collapsed artery. A key observation is that measuring the progress of the opening of the vessel provides a measurement on the displacement of elementary blood volume dV (20).

Following the principle discussed above, preferred embodiments of the apparatus for non-invasive monitoring of heart performance in accordance with the invention include the following main components:

1. A device for producing the collapse of an artery on the arm or wrist, based on applying external pressure. The exerted pressure must be sufficient to produce the collapse but not too high to prevent substantial disruption in the normal flow of blood and infliction of discomfort to the patient.

2. A sensor for measuring the change of volume occurring when the blood flow opens the artery. The sensing device should be such that it follows the progress of the elementary volume in the artery.

3. A processing unit for recording, analyzing and preferably also storing the data retrieved from the sensor.

Ideally, the sensor sensing the change of volume should be long enough to sense the progress of the elementary blood volume (22) during the whole period from rest to rest. However, this would require the application of a uniform external pressure and consequent artery collapse over a long section of the artery, which is not practical for a non invasive—portable device. Instead, one could use a shorter sensor for direct measurement of the volume change, and extrapolate the data to cover the whole blood flow period by using an appropriate algorithm. This implementation suffers from the difficulty that it still requires a relatively long sensor, which introduces non-linear modifications to the sensed signal that are difficult be compensated for. For instance, if an inflatable cuff is used, then the flexible nature of the cuff combined with a relative large size introduces deformations that affect the measurements and cannot be disregarded.

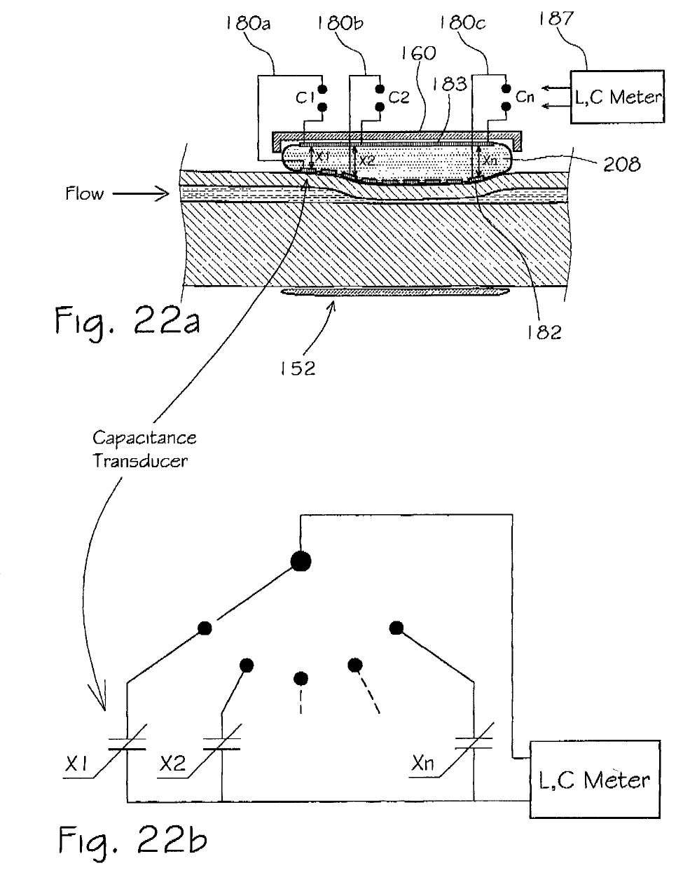

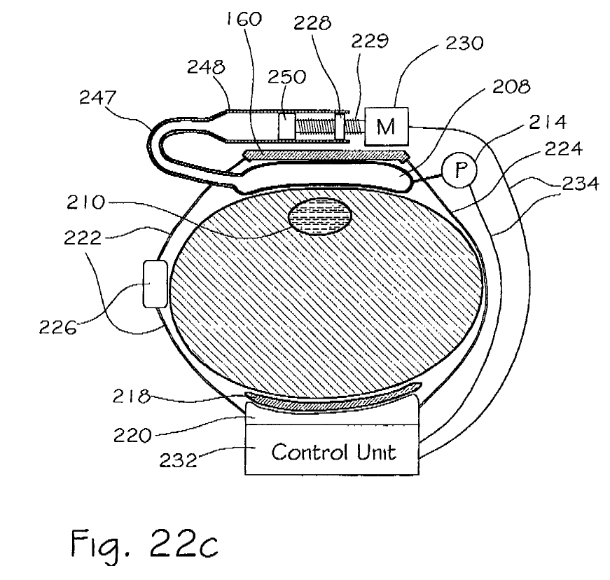

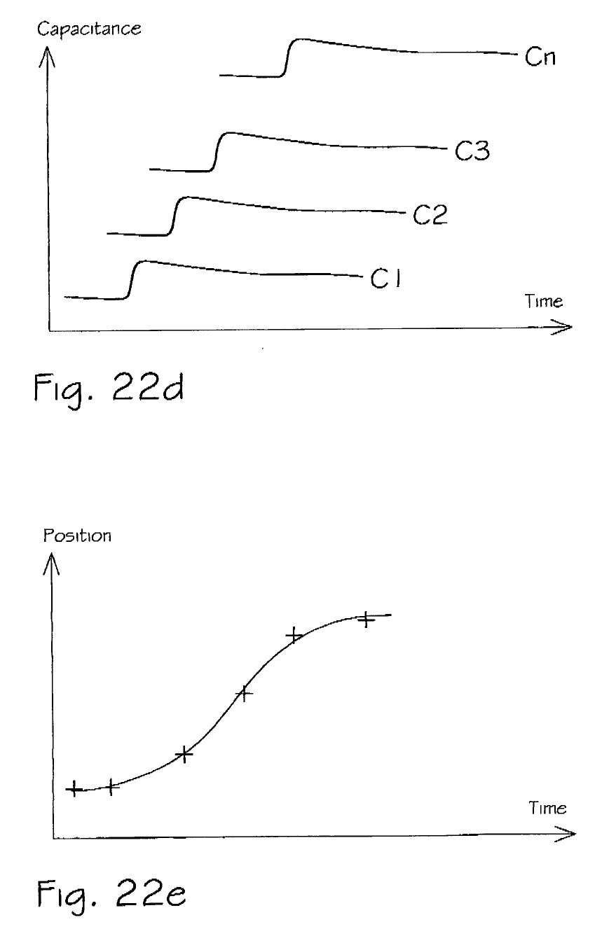

In one of the novel aspects of the invention disclosed hereinafter, the sensing unit uses a plurality of relatively short and simple sensors, and a numerical algorithm is used to combine the information from the plurality of sensors in a way that drastically increases the reliability, sensitivity and signal to noise ratio of the device. The combination of the plurality of sensors in a way that drastically increases the reliability, sensitivity and signal to noise ratio of the device. The combination of the plurality of sensors then allows for an adequate extrapolation of the signal to cover the displacement of the elementary volume between two diastoles. The resulting invention still allows the measurement of the peripheral velocity time integral (and consequently the mean velocity), and hence provides much more comprehensive information than a sensor measuring instantaneous velocity alone. In alternative configurations, a single transversal deformation sensor may be used for providing a linear and reliable measurement of the progress of the opening of the artery (for example, FIG. 22). Deformation may be measured in many ways, some of which include using piezoelectric transducers, optical sensors (with lasers, optical fibers etc.), capacitors, and other types of sensors reacting to deformation.

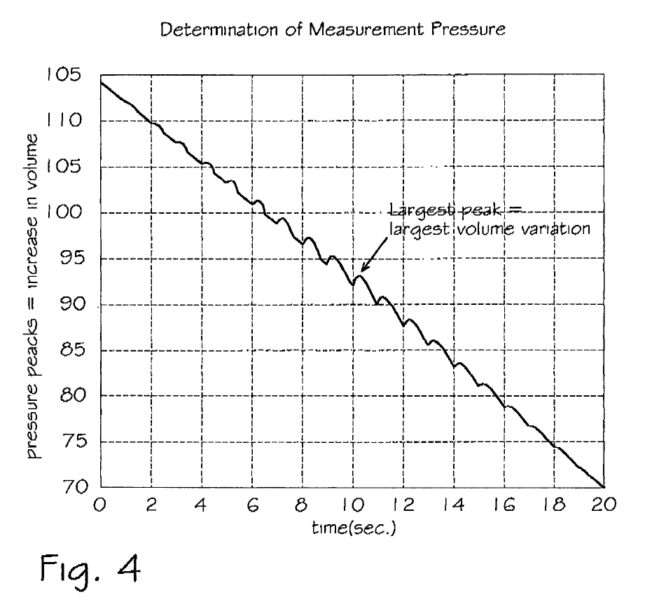

The external pressure to be used during the measurement should be just enough to collapse the artery in the diastolic phase so a slight increase in the internal blood pressure is sufficient to recover the artery from collapse to fully opened (FIG. 2). In preferred embodiments of the present invention this pressure is not determined a priori but rather obtained via an indirect measurement, which can be described as follows. First, a relatively large external pressure is applied to assure that the artery collapses in the region of the external pressure. Subsequently, the pressure is reduced gradually while monitoring changes in the volume. During the reduction, at first no change in volume is observed since the internal blood pressure is not sufficient to open the artery at any phase of the heart cycle, but then at some point the artery opens and closes periodically following the pressure variations. As a result, one observes a graph as shown in FIG. 4.

The pulses in the pressure curve in FIG. 4 are proportional to the change in volume following the progress of the elementary volume. The largest pulse amplitude represents the point at which the external pressure is large enough to collapse the artery, but a small increase in the internal blood pressure immediately causes the complete opening of the vessel. Consequently, the pressure for taking the measurement can be roughly determined by applying a monotonically decreasing external pressure and then computing by means of a numerical algorithm the pressure at which the largest variation occurs. More detailed algorithms for the a-priory determination of the measurement point are disclosed herein below.

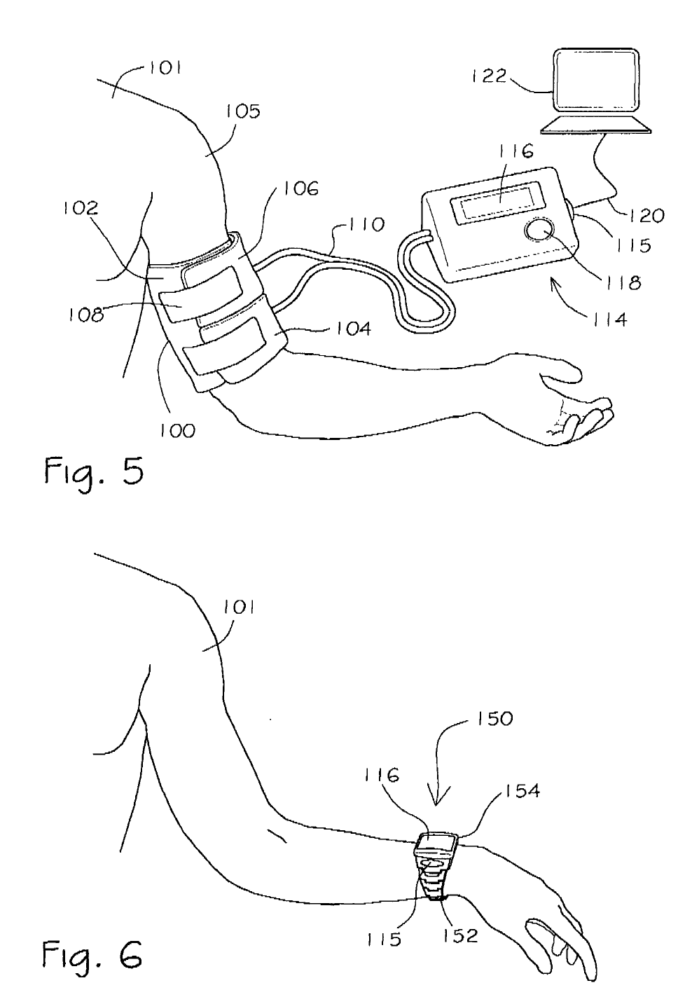

Reference is now made to FIGS. 5 and 6 illustrating noninvasive devices for monitoring heart mechanical performance in accordance with two preferred embodiments of the present invention, worn on the upper arm (FIG. 5) or on the wrist (FIG. 6).

In a preferred embodiment of the present invention, a monitoring device is worn by a patient on the arm (FIG. 5) in the shape of a cuff 100. The cuff is designed to facilitate the collapse of a blood vessel (typically an artery) within the arm and measure the incremental opening of the artery following the progress of blood through the vessel.

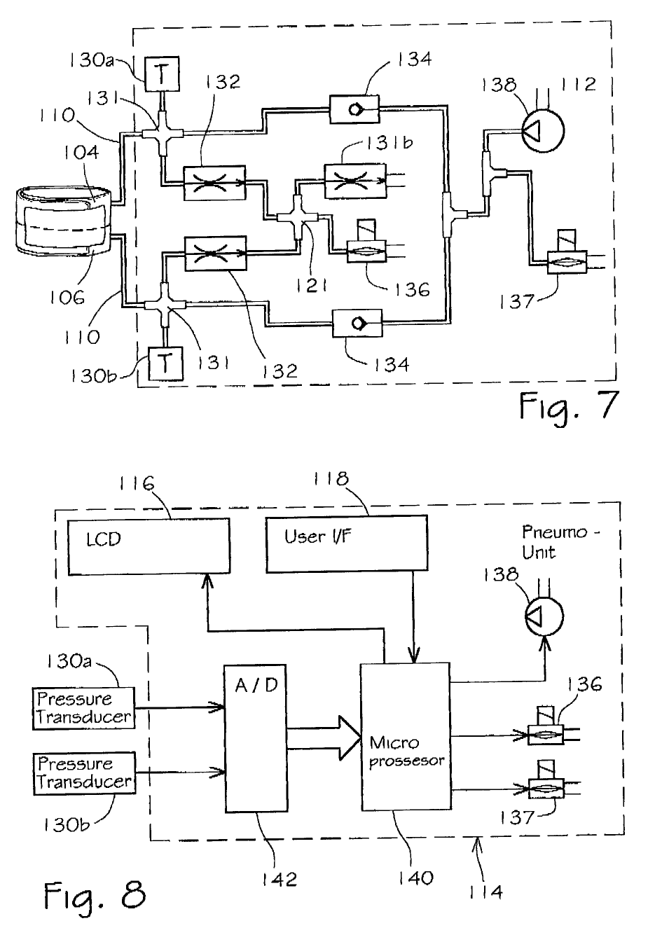

In the particular embodiment of FIG. 5 the cuff 100 comprises two adjacent inflatable segments 104, 106, that upon inflation exert, each, a pressure on the arm enough to collapse a blood vessel located under the cuff. However, in other similar embodiments more than two segments can be used. The cuff may be secured over the arm by fastening bands 108 (similarly to blood pressure measuring cuffs). Each inflatable segment coupled to a pressure sensor (not shown in FIG. 5 but schematically represented in FIG. 7, as 130a and 130b) that is separately connected to the control unit 114, a housed analyzer having readout display 116, optional user interface 118 and optional output socket 115 allowing the device to be connected to an external computer 122 via cord 120. The connection lines 110 may be pipes (if the pressure sensors are located in the reader—in order to transfer the pressure experienced within each inflatable segment to the sensors) or electric conductors (if the pressure sensors are positioned within the cuff and output electric signals). Control unit 114 includes electronic or pneumatic components as is described herein after. Note that throughout the present specification and claims by “sensor” is meant not only a single sensor but also a number of sensors or sensing means.

Referring now to FIG. 6, in another preferred embodiment of the present invention the monitoring device and the analyzer are incorporated in a device worn by the patient on the wrist in the shape of a bracelet 150, secured to the wrist by strap 152. The bracelet contains an inflatable or mechanical mechanism for applying a gradually reducing pressure, as in the case of the first embodiment. Housing 154 houses the electronic and mechanical components of the device, as explained hereinafter.

Comparing the embodiments disclosed in FIG. 5 and FIG. 6, it is noted that while the artery in the upper arm passes deep inside the arm, in the wrist the artery is located close to the surface of the limb, which is reflected in the respective mechanical embodiments. In the upper arm measurements are taken in circumferential aspect, using a cuff surrounding the arm circumferentially as shown in FIG. 5. For the wrist apparatus shown in FIG. 6 it is sufficient to focus on a small area adjacent to the radial artery, although it is also possible to use a device applying circumferential pressure. Both embodiments include an inflatable or mechanical mechanism for applying sufficient pressure to assure a complete collapse of the respective artery, and subsequently decreasing the pressure, to allow for a gradual and progressive opening of same artery.

Referring back to FIG. 5 and FIG. 7, the operation of the arm-mounted embodiment of the present invention is described herein below with further details. The inflatable cuff 100 is divided into two independent inflatable segments 104 and 106. These segments have roughly the same size, and are located at a fixed predetermined distance between them. Each segment is communicating with a sensor (130a,b) for measuring the instantaneous pressure within the segment. In a preferred embodiment, typically this sensor is a micro machined membrane piezo-resistive transducer, such as the sensor manufactured by Motorola™ and marketed under the brand name of MTX2201, although other transducers of air pressure could also be used.

Pneumatic arrangement is provided which keeps substantially the same static pressure in the two segments throughout the measurement. The pneumatic arrangement of this embodiment is illustrated in FIG. 7.

A full cycle of the pneumatic components would look like this: With valves 136 and 137 closed, the air pump 138 pumps air to the inflatable cuff segments 104, 106. When the desired high pressure is reached, the pump stops working. The high pressure is typically above the patience's systolic pressure. Then, valve 137 is opened, so that the pressure to the right (respectful of the drawing) of the non-return, one-way, valves 134 drops and prevents the air from flowing back. The air flows from each segment of the cuff through the pressure regulators 132, and then through the regulator 132b. While the air is flowing from the cuff, the pressure transducers 130a and 130b measure the internal pressure in each section of the cuff. When the internal pressure of the cuff drops well below the diastolic pressure, measurements are stopped. Then, valve 136 is opened to allow the remaining air to exit the cuff. At this point a new cycle may begin.

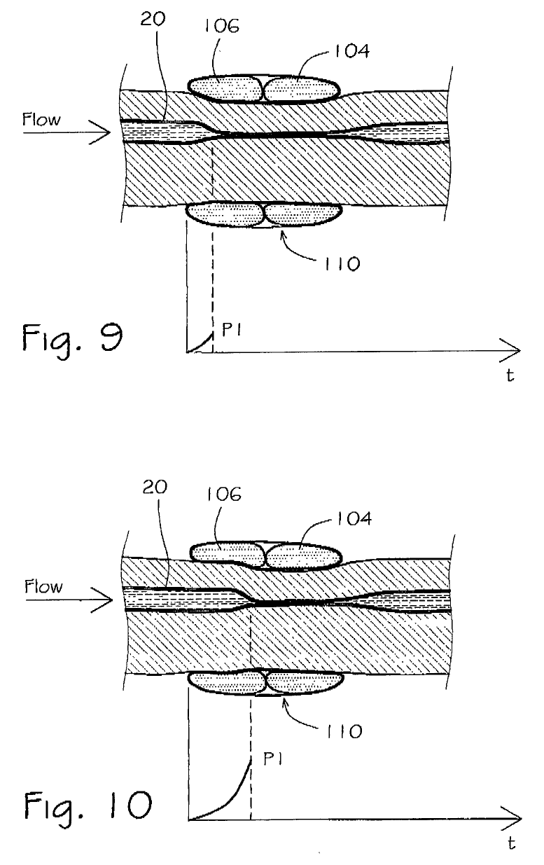

The electronic circuit of a preferred embodiment of the present invention is illustrated in FIG. 8. The analog measurement of the pressure sensors 130a and 130b are digitized by an A/D converter 142. The resulting data is processed by micro-processor 140 using the algorithm described herein below. The results of the calculations are shown to the user on display 116 (for example LCD). The user may input required data or commands using interface (such as keyboard) 118. The micro-processor 140 also controls the pneumatic circuit, by operating the air pump 138 and closing and opening the solenoid valves 136, 137. Measurement results may be stored in memory unit 143.

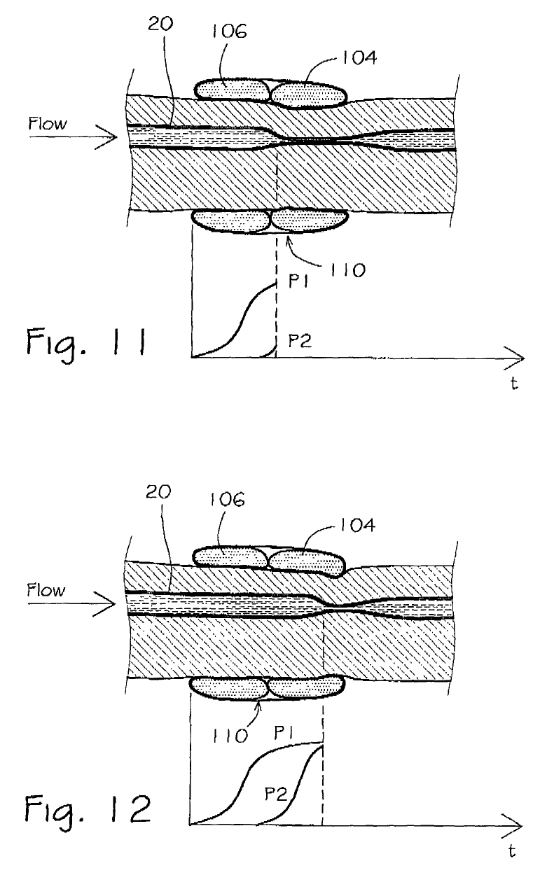

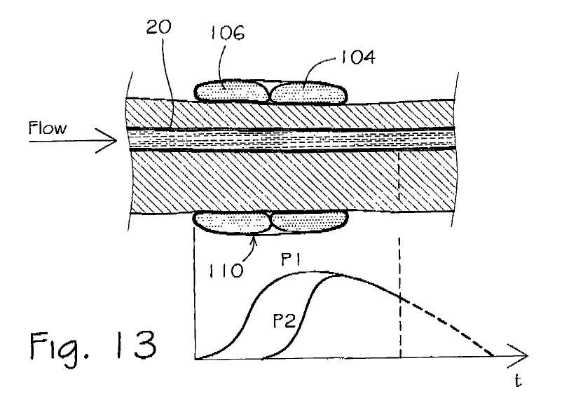

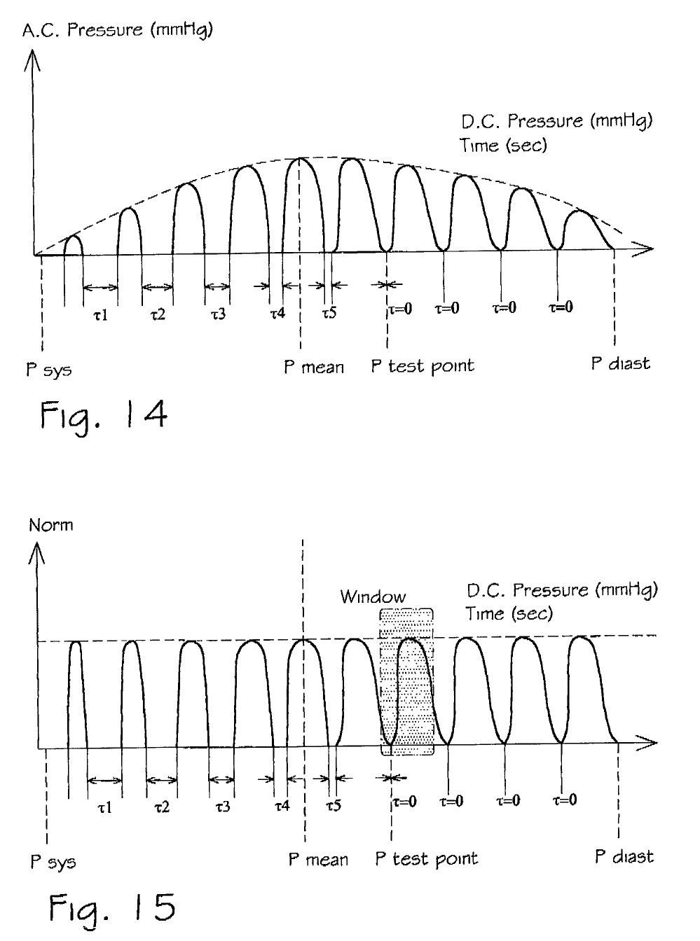

To better understand the algorithm for computing blood flow, consider the sequence of FIGS. 9 to 13. The figures show the progress of the elementary blood volume flow through the artery as the blood is pushed forward during the heart cycle. This progress results in an increase of the cross section under the cuffs and consequent increase of the pressure within the cuff segments, which can be sensed by the sensors 130a and 130b (see FIG. 7 and FIG. 8). The corresponding pressure profiles P1 and P2 are digitized by the A/D converter 142 and input to the micro-processor. The full cycle of pressure increase and decrease shown in FIG. 13 corresponds to a diastole to diastole cycle. An example of the measurements obtained by one sensor (e.g. 130b) over the entire measurement is shown in FIG. 4, and in FIG. 14 after the quasi-static pressure of the cuff has been subtracted as described herein below. By quasi-static pressure is meant the pressure due to the inflatable device, which reduces gradually as the air exits the pneumatic circuit. Note that the signal from the more distal sensor is delayed with respect to the proximal sensor, by a quantity roughly proportional to the distance between the segments divided by the instantaneous velocity of the blood flow. Note, though, that the present embodiment determines not just the delay between the proximal and distal pulses but also the integral of the elementary blood element propagation velocity over a heart cycle as described herein below.

While the quasi-static pressure in the cuffs is reduced from above systolic to below diastolic pressures, the pressure inside each of the segments of the sensor is measured and stored in memory. After all the pressure data has been collected, an algorithm is used to determine the pulse or set of pulses, corresponding each to one heart cycle, to be analyzed for the purpose of deducing hemodynamic parameters.

The algorithm as applied in the embodiment of FIG. 5 comprises the following steps:

1) Calculating the instantaneous pressure changes within each segment of the cuff. This is carried out by subtracting from the pressure data of each segment the quasi static pressure. This can be done, for example by approximating the quasi-static pressure as a function of time using a low order polynomial fit to the pressure data, or by smoothing of the measurements data using appropriate low pass filter. The subtraction of the quasi-static pressure results in a time data containing instantaneous variations in internal pressure following the opening of the arteries. FIG. 14 shows a typical example of the resulting data.

2) The data for each one of the sensor segments is divided to heart cycles. At each one of these periods, the local maxima and minima are computed, and the data is normalized between 0 (corresponding to the minimum at each period) and 1 (corresponding to the maximum at each period). In the preferred embodiment of the algorithm, two low order splines fit the maxima and the minima. The data is then adjusted using these splines to be normalized between 0 and 1. FIG. 15 shows a typical example of the resulting data.

3) Using the resulting nominal data history for one of the sensors (in the preferred embodiment, the proximal sensor), a search for the correct test pressure along the pressure curve is performed. By test pressure is meant the quasi-static pressure at which the measurement data is analyzed to deduce the hemodynamic parameters of interest. As shown in FIG. 15, at high pressures there exists a measurable time separation between the falling and raising edges of subsequent pressure pulses, due to the fact that the artery is collapsed during a part of the cycle. See the time periods τ₁ to τ₅ in FIG. 15. As the external pressure is reduced, the artery remains collapsed less time and therefore the time separation between consecutive pulses also reduces. In FIG. 15 this is illustrated by the fact that τ₁>τ₂>. . . >τ₅. At one point, denoted by “P test point” in FIG. 15, the pulses appear one after the other with no noticeable time separation between them. The quasi-static pressure at which this phenomenon is first observed is the desired test pressure. At this point, the external pressure is just sufficient to collapse the artery momentarily at the diastole but the artery re-opens and allows blood propagation as soon as the next pressure pulse from the heart starts building up. In the presence of noise, the preferred embodiment implements the above step by measuring the separation time between pulses at a pre-specified threshold. The resulting values first decrease as pressure decreases, and eventually flat out close to zero. The test pressure is determined as the interception point of curves fitted separately to the high pressure and low pressure data.

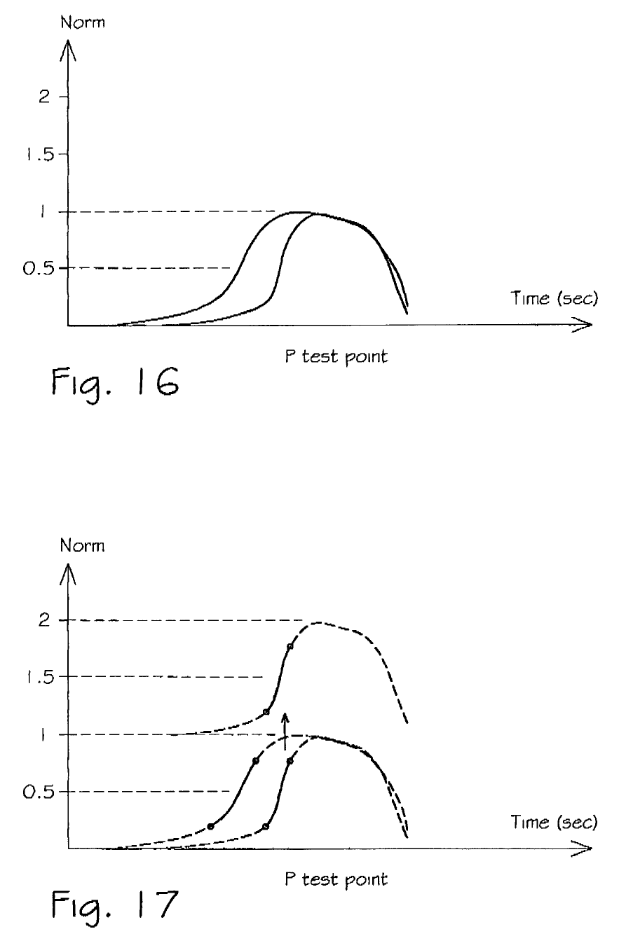

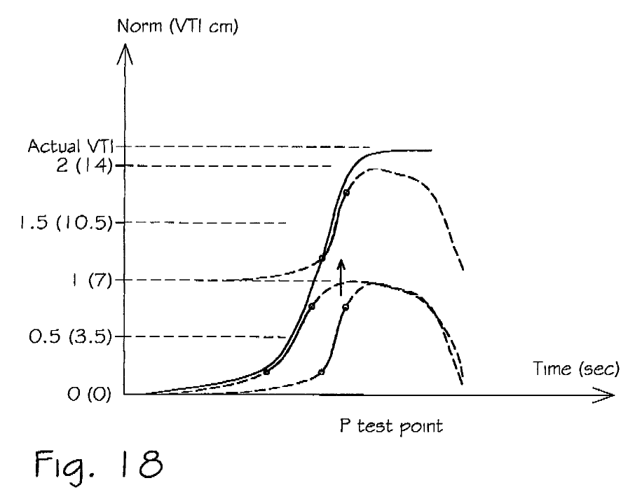

4) Once the test pressure and corresponding heart cycle have been determined, a time window is define to separate the data of one pulse as shown in FIG. 15. The pressure variations in that window for both cuff segments are process together as shown in FIG. 16. The signals in FIG. 16 represent the build up of pressure in the cuff segments, corresponding to volume in the blood vessel. Hence, the vertical axis in the graph corresponds to propagation of the blood along the artery length. Because of the flexible material of the cuff segments, the measurements at the edges of the cuff are highly nonlinear as reflected at the leading edge and close to saturation of the pulses. Therefore, only the central section of the raising edge of each pulse is used for the computations. For instance, if the whole pulse is normalized between 0 and 1 as shown in FIG. 16, only the sections of the plots with values between typically 0.2 and 0.8, are processed. Considering now that the cuff segments length each corresponds to full scale 0-1 in FIG. 16 and the distance between the segment equals the length of each segment, the data from the distal sensor is shifted up by one vertical unit relative to the data of the proximal sensor, corresponding to one segment length. This is illustrated in FIG. 17. The combined data for both sensors represent sections of the artery volume—time curve.

5) The data in FIG. 17 is fitted to a theoretical curve approximating the volume increase during the diastolic to systolic transition, as shown in FIG. 18. For example, the inventors have found that a Sigmoid function

Y=δ+k/(1+e^{-γ(x-C)})

provides a proper fit with consistent results. Here, k is proportional to the PVTI, which is essentially the saturation value of the function, and δ, γ and C are adjustment constants.

6) The inventors have found that it may be advantageous to analyze results for a number of pulses below and above the test pressure. Typically up to ±5 pulses are used. The PVTI value for the desired test pressure is determined by a polynomial fit of the PVTI values above and below the test pressure. This procedure reduces sensitivity to noise and improves accuracy while still providing meaningful clinical results.

An alternative algorithm replacing steps 4-5 above with somewhat less accurate results is described next.

1) The data is processed and the desired test pressure is determined as described in steps 1-3 herein above.

2) The propagation time of the elementary blood element from the proximal to the distal section is given by the time difference between the two curves shown in FIG. 16. Hence, the average propagation time is approximated by the average of the time difference between the curves above and below given thresholds. The inventors have used the thresholds range of 0.2-0.8 for performing the calculations on clinical data.

3) The average propagation velocity, approximating the PVTI, is the distance between the cuff sections centers divided by the average propagation time.

The detailed algorithms are provided herein above by a way of examples. The reader experienced in the art will appreciate that other algorithms can be used to analyze the measurement data and extract the hemodynamic parameters within the scope of this invention.

In addition to the Peripheral Time Velocity integral (PVTI), the apparatus described above measures the heart pulse rate HR and can measure the systolic, mean and diastolic blood pressures using the pressure data from either one of the sensors and algorithms well known in the art and used in many commercial instruments. Using these results and assuming the non-collapsed artery cross section AS is substantially constant over time, the following hemodynamic parameters can be calculated:

The peripheral stroke volume is:

PSV=AS×PVTI

The peripheral cardiac output is:

PCO=PSV×HR

The peripheral cardiac work per cycle is:

PCW=PSV×MAP

Here, MAP denotes the mean artery pressure.

While the absolute values for these parameters cannot be determined from the PVTI data, as the arterial cross section AS is not known, there is still advantage to calculate the relative value of these and other parameters for the purpose of diagnosing the heart and vascular system condition.

As a feasibility study to the device described in the embodiment of FIG. 5, a series of measurements were taken to 44 different patients as a proof-of-concept for the above apparatus. The measurements were made at the Coronary Unit of the Sieff Government Hospital, Safed, Israel, between December 2001 and May 2002. The study involved application of Tc-99m Sestamibi SPECT with pharmacological effort induced by dobutamine, which is the gold standard for detection of myocardia ischemia. When the PVTI results obtained by the inventive apparatus were compared to the results obtained by the Tc-99m Sestamibi SPECT, the PVTI test identified as positive 8 of the 11 patients identified as positive by the Tc-99m Sestamibi SPECT. Moreover, from the 33 patients identified as negative by Tc-99m Sestamibi SPECT, 30 were identified as negative by the PVTI criterion. These results correspond to overall sensitivity (TP/(TP+FN)) in using PVTI as compared to Tc-99m Sestamibi SPECT technique of 73%, and specificity (TN/TN+FP) of 91%.

Other preferred embodiments of the present invention refer to wrist-mounted monitoring devices such as shown in FIG. 6. Wrist mounted devices can be based on the same configurations and principles as the upper arm mounted devices described herein above. However, advantage can be made of the proximity of the radial artery to the skin surface to apply the external pressure only locally rather than circumferentially.

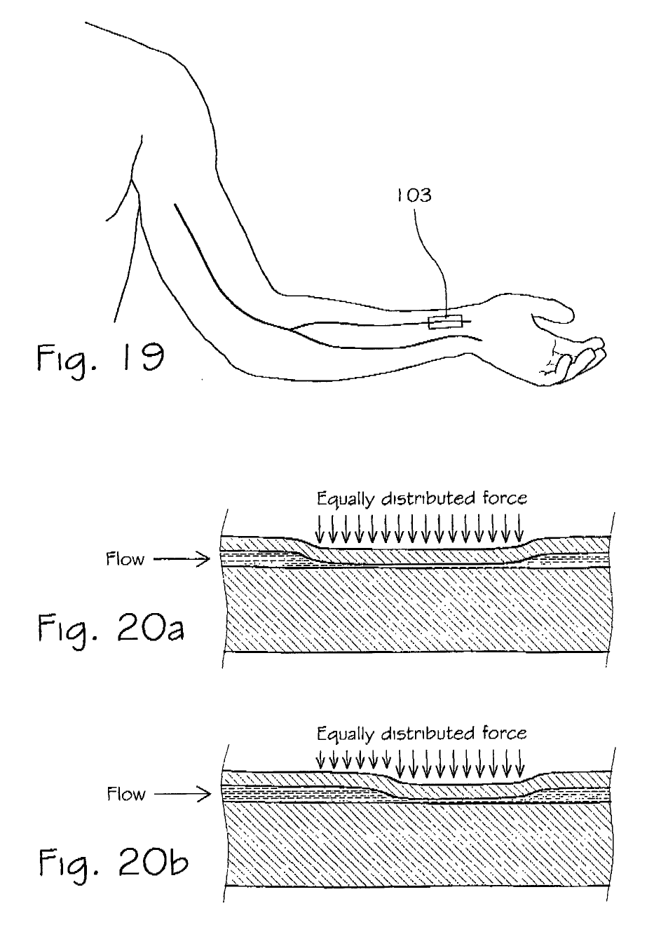

For understanding a preferred embodiment of a monitoring device mounted on the wrist reference is made to FIG. 19 showing that the artery on which the measurement is performed is preferably the radial artery. FIG. 19 shows the region 103 on which the external pressure is applied on the wrist.

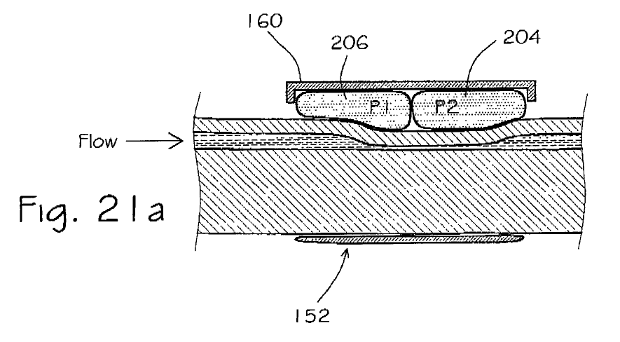

FIGS. 20a, 20b show a transversal cross-section of the wrist under this location. An arrow indicates the direction on which the blood flows. The figures do not show the strap or band required for applying external pressure as explained herein above. The strap or band are coupled to a mechanism, for example inflatable, for applying a external pressure. Alternatively the strap can be mechanically tightened, for example by means of a motorized rotor spinning strings about it thus shortening the strap, and reversing the direction of spin to loosen the strap. Other pressure applying straps can also be used.

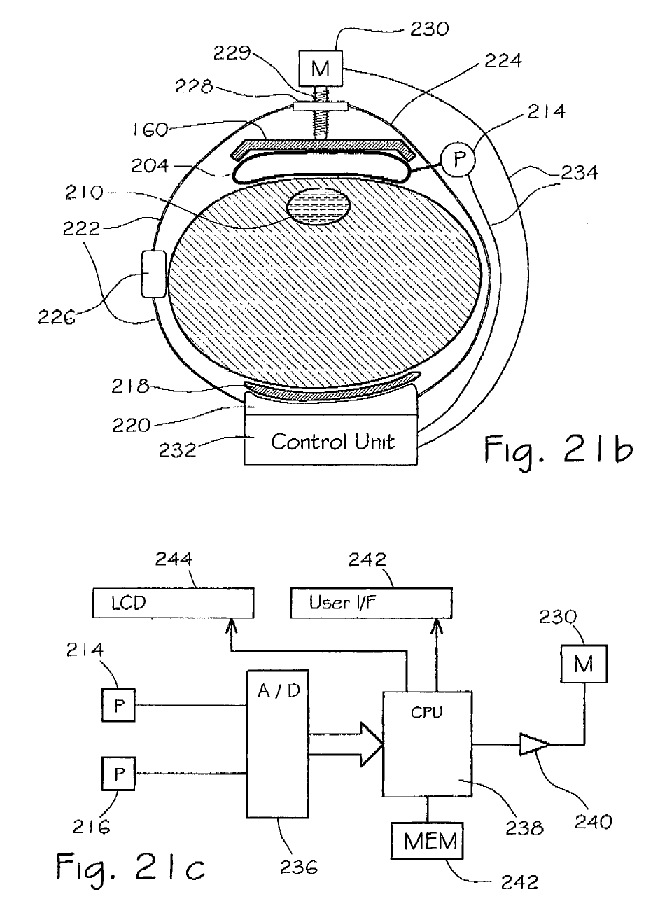

The apparatus applies an equally distributed force (pressure) on the region of interest (see FIG. 20a). The pressure is sufficiently high to produce the collapse of the artery, at least momentarily during the diastolic phase. This is illustrated in FIG. 20b, where the blood flow increases the internal pressure and opens the artery. It is important that the external pressure shall be equal on all points as can be achieved by means of a pneumatic device, similar to the cuff shown in FIG. 5, or by an other mechanical structures (see FIGS. 21-26).

FIG. 21a shows a pressure applying structure of two cushions (204, 206) coupled to a bridge 160, the cushions are inflatable as in the embodiment of FIG. 5, and provided with a pneumatic and control systems as shown in FIGS. 7, 8. Bridge 160 is secured to place by strap 152 surrounding the wrist and provide counter-pressure when the cushions are inflated. The pneumatic and control system may be mounted in a separate unit or attached to the wrist mounted device providing a fully mobile battery operated device. The operation and data analysis of this device is identical to the embodiment described herein above for the upper arm.

FIG. 21b shows another embodiment of the present invention, where the cushions are not air-inflatable but rather filled with fluid. The Figure shows an axial cut with the radial artery 210 facing up. Cushion 204 is mounted underneath bridge 160 and provided with a pressure sensor 214. Cushion 206 and corresponding sensor 216 are not shown. On the opposite side of the arm, cushion 218 and bridge 220 are positioned. Straps 222 and 224 linked to the bridge form a bracelet around the wrist. The straps are adjustable in length to fit to any particular patient and are provided with a latch 226 to allow mounting the apparatus on and off the wrist. The adjustment to a particular patient may be manual or automatic using the drive system and control unit as described below. Straps 222, 224 are connected to a nut 228, mounted on lead screw 229, which when turned by motor 230 it applies pressure on bridge 160. Motor 230 may be mounted on bridge 160 using a bracket (not shown). The motor and pressure sensors are connected to control unit 232, mounted on bridge 220, by electrical wires 234.

FIG. 21c shows the details of a proposed control unit 232. The unit houses an A/D converter 236, which transfers the pressure data from sensors 204,206 to CPU 238. The CPU also controls motor 230 via driver circuit 240, responding to commands from user I/F 242 and displaying results by display unit 244. Measurement results may be stored in memory unit 242. Power for the operation of the device is provided from batteries or other power source (not shown).

The function of the straps and motor elements is to increase or decrease the external pressure applied by cushions 204,206 onto the artery 210. In any other way, the operation of the device is identical to the other embodiments described herein above.

The reader will appreciate that many other mechanical arrangements can be used to apply the external pressure by reducing the circumference of the bracelet and the embodiment shown in FIG. 21b is disclosed only by a way of example.

FIG. 22a shows a different embodiment whereby the external pressure is applied by using a rigid bridge (160) for support and one cushion (208), inflatable or filled with hydraulic fluid, that applies the pressure to the limb. Cushion 208 is provided with deformation sensors comprising of an array of capacitor plates, one common plate and an array of opposing plates (alternatively is may be possible to provide an array of pairs of plates), wherein the mechanical changes are determined by measuring changes in the capacitance of the capacitors, due to changes in the distance between an upper side of the cushion, and sectors of a lower side of the cushion. Using an LC meter 187, the capacitance is measured between plate 183, which is held rigidly by bridge 160, and each separate plate 182. By measuring the capacitance of each capacitor, the deformation of the sensor is determined. For better understanding of the embodiment, FIG. 22b shows an equivalent circuit of the deformation sensor. The deformation variation tracks the opening of the artery and hence the progress of the blood movement while the artery recovers from collapse.

FIG. 22c shows the details of a mechanism for applying external pressure in an embodiment based on the structure disclosed in FIG. 22a or other embodiments of the present invention, where the cushion is filled with hydraulic fluid material. The Figure shows an axial cut with the radial artery 210 facing up. Cushion 208 is mounted underneath bridge 160 and provided with a pressure sensor 214. On the opposite side of the arm, cushion 218 and bridge 220 are positioned. Straps 222 and 224 linked to the bridge to form a bracelet around the wrist. The straps are adjustable in length to fit to any particular patient and are provided with a latch 226 to allow mounting the apparatus on and off the wrist. Tube 247 connects the cushion 208 to cylinder 248 fitted with piston 250 which can by used to increase or decrease the fluid pressure in cushion 208. The piston is driven by lead screw 229, which is driven by motor 230. Motor 230 is mounted on bridge 160 using a bracket (not shown). The motor and pressure sensor 214 are connected to control unit 232, mounted on bridge 220, by electrical wires 234. Control unit 230 may be similar in its structure and operation to the unit disclosed in FIG. 21c, however, with the added function of acquiring and analyzing the data from the capacitive sensor.

The analysis of the data form the preferred embodiment is described next.

1) The desired test pressure is found from the pressure data as described herein above.

2) The acquired capacitance-time curves (typical curves are shown in FIG. 22d) for each of the capacitor sections are analyzed to determine the time of the pulse. One way to define the time is the crossing point of the leading edge of the pulse and a pre-set threshold. A more elaborated way is to determine the time at which the leading edge reaches a pre-set fraction of the pulse amplitude. This determined time corresponds to the time at which the blood entering the artery while opening the collapse reaches the specific transducer.

3) A graph of pulse time versus capacitor position is generated where the position relative to the first transducer (most proximal) is known from the structure of the apparatus. See FIG. 22e.

4) The data is fitted to the empirical curve provided herein above for the pressure sensors data (FIG. 18). The Peripheral Velocity Time Integral PVTI is determined as the saturation value of the fitted curve.

The procedure may be repeated and results calculated for several pulses (typically 5) above and below the optimal test pressure. The PVTI value for the desired test pressure is determined by a polynomial fit of the PVTI values above and below the test pressure.

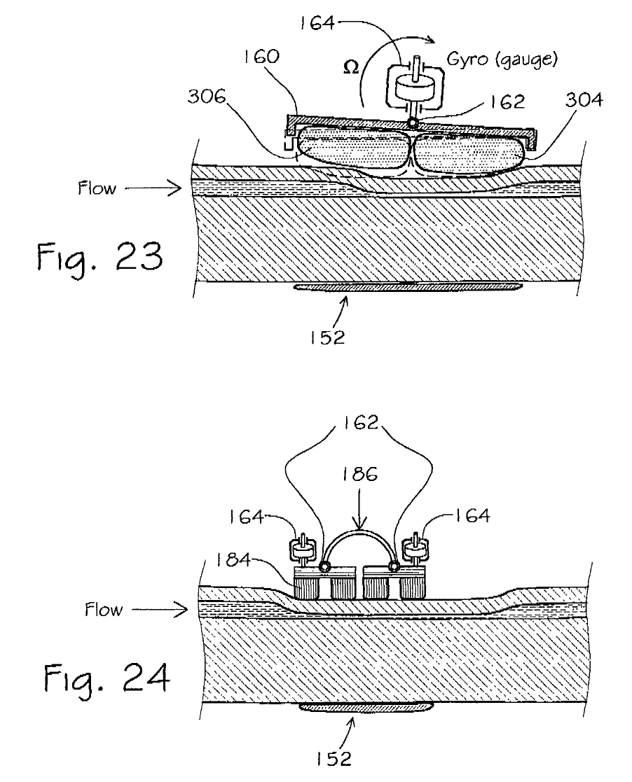

In an alternative preferred embodiment illustrated in FIG. 23, the mechanical changes due to the opening of the artery are sensed by applying pressure using cushions 304 and 306. These cushions are connected to a rigid bridge 160 in such a way that pressure applied to bridge 160 will be transmitted uniformly to cushions 304, 306. A solid state (or other type) gyroscope 164 is mounted on the bridge for measuring the angular velocity about the pivot 162. The gyroscope 164 may be, for example, the gyroscope marketed by MuRata™ under the name ENC-03G. The flex pivot 162 is arranged so that the center of rotation of the pivot lies above the radial artery, to assure that a rotation motion of bridge 160 will be created by the flow of blood.

While a device with two cushions as shown in FIG. 23 meets the goals of the invention, it is possible to increase sensitivity and accuracy of the measurement by using more than one pair of cushions as shown in FIG. 24. Cushions 184 are coupled in pairs to plurality of bridges 160, which are in turn connected to a top bridge 186 that transmits external pressure uniformly to the lower side of the cushions and allows for rotational motion of the bridges 160. The pressure is applied to top bridge 186 by a device similar to FIG. 21b or by another mechanical arrangement. The joining device 186 is attached to the bridges by means of bearings or by flex pivots 162 to allow the appropriate transmission of the motion. The angular motion of sensors 164 provides a measurement of the progress of the opening of the artery as explained inhere below.

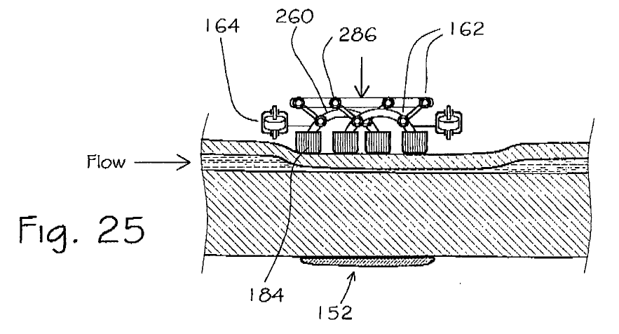

Alternatively, two bridges 260 can be interlaced as shown in FIG. 25. This gives rise to a slightly more complicated mechanical configuration but allows a larger gap between the cushions of each bride, thus amplifying the angular motion. Bridges are connected together by a top bridge 286 so that the pressure is transmitted uniformly to the down side of the cushions 184. Connections between the moving parts are made by bearing or flex-pivots to allow angular motions while preventing linear motions. Note that the gyroscopic sensor is physically coupled to the bridges 260 (the graphical representation of the gyroscopes in FIG. 25 appears to be floating over the cushion for clarity only).

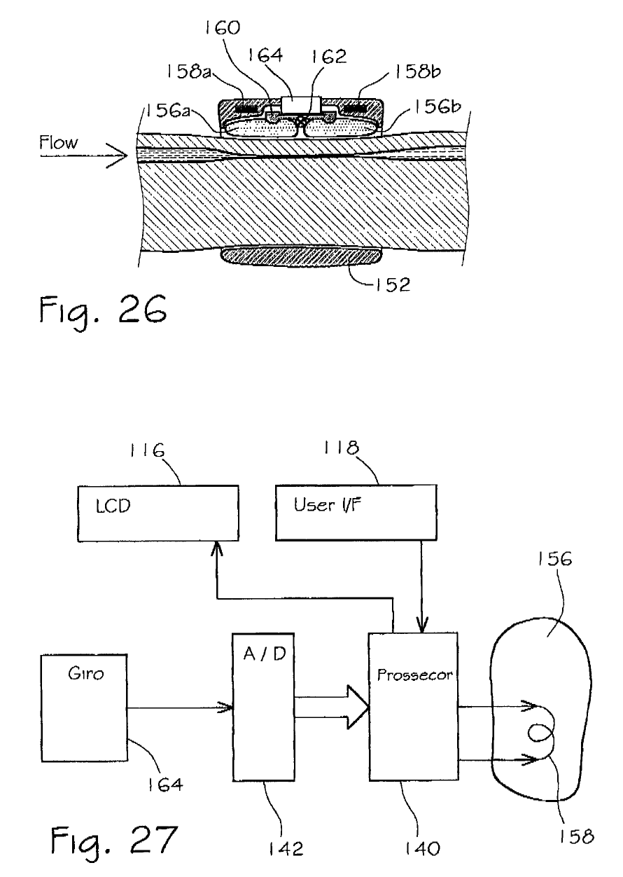

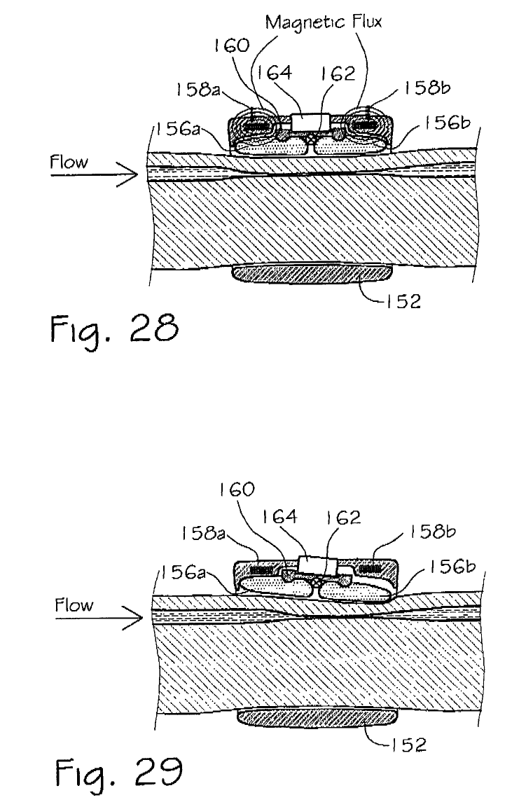

Another preferred embodiment is illustrated in FIG. 26. A bracelet 152 is provided surrounding the wrist. The upper part of the bracelet contains the measuring and electronic components. The measuring sensor consists of two cushions 156a and 156b filled with ferromagnetic fluid, such as ferromagnetic fluid that is composed of base liquid, ferromagnetic particles and chemically adsorbed surfactant, and may be obtained from Sigma Hi-Chemical Inc., of Japan. This ferromagnetic material has the property of being fluid in the absence of a substantial magnetic field and becoming solid when a magnetic field is present. Electromagnet coils 158a and 158b are provided in the vicinity of each cushion to induce a magnetic field when a current is passed through these coils. The sensor further includes a bridge 160 pivoting about a pivot 162.

The functioning of this embodiment and the preceding embodiments using rigid cushions can be better understood by referring to FIGS. 26 to 29. FIG. 26 shows the state of the sensor when a measurement has not been taken yet. In the absence of a magnetic field, the ferromagnetic material is in a fluidic state and hence the cushions adapt to the shape of the wrist. As external pressure is applied, the artery reaches collapsed state and blood flow stops. The external pressure is applied by a mechanical structure coupled to bracelet 152 under control of microprocessor 140 (FIG. 27).

Once at sufficiently high external pressure, microprocessor 140 generates a signal that excites the electromagnetic coil 158a and 158b thus hardening the ferromagnetic material within the cushions 156a and 156b (FIG. 28). The external pressure is than reduced monotonically as done in the previous embodiments to the region where the internal blood pressure is sufficient to open the blood vessel. Since the cushions are now rigid, they do not comply anymore with the blood flow, and that the progress of the unit volume of blood through the artery can be tracked. The unit volume of blood first raises the left leg of the bridge 160 respective to FIG. 29 thus causing a clockwise rotation of the bridge. Then, as the blood flow progresses the bridge rotates in the opposite sense. The gyroscope 164 senses these rotations. The A/D converter 142 samples the gyroscope measurements and inputs the digital signal into the microprocessor 140.

The analysis gyroscope output data in the embodiments shown in FIGS. 23-26 is described next. The microprocessor 140 transforms the rotational velocity into a measurement of the progress of the unit volume of blood through the artery, using the formulas:

V(t)≈(c+h/θ²)\dot{θ}, \dot{θ}≧0

V(t)≈−(c+h/θ²)\dot{θ}, \dot{θ}<0

In this formulas, V(t) represents the blood velocity and \dot{θ} is the angular velocity of the bridge as measured by the gyroscope 164. Plotting the results as a function of reducing pressure, a curve similar to FIG. 14 is obtained and is used to determine the desired test point where is artery is collapsed only momentarily during every diastole. The microprocessor 140 integrates the velocity curve for that pulse to compute the VTI.

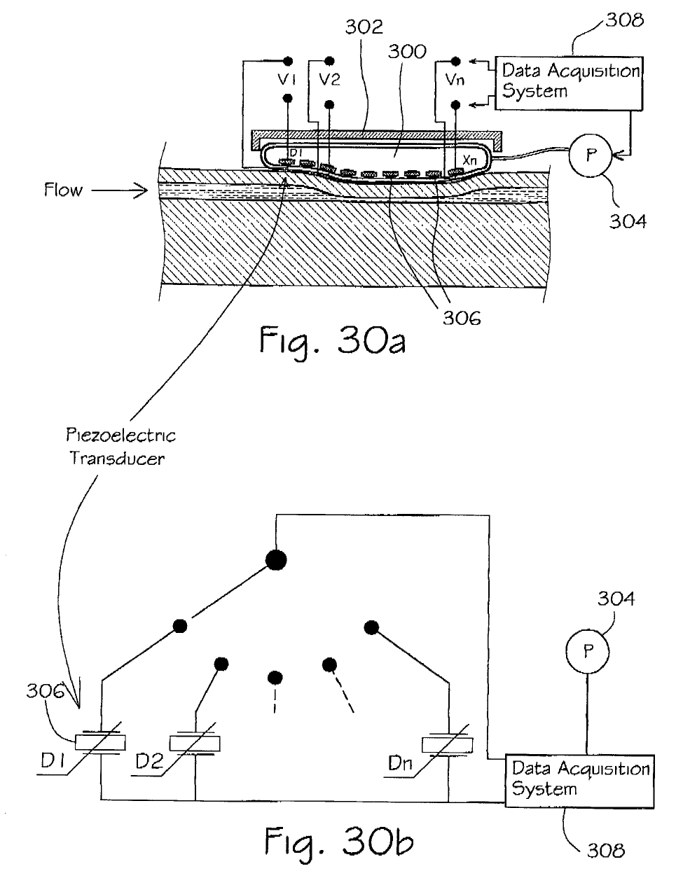

Reference is now made to FIG. 30a, showing a schematic structure of another preferred embodiment of the present invention. A single cushion 300 filed with fluid is pressed against the limb with a bridge 302 which in turn is provided with means to apply and reduce the external pressure (not shown). In alternative embodiments the cushion may be replaced by inflatable cuff. The cushion or cuff are provided with pressure sensor 304 and array of piezoelectric transducers 306 responsive to deformation mounted along the artery to be monitored. Typically 6-10 transducers are used but a smaller or larger number is possible as well. Data acquisition system 308 is provided (FIG. 30b) to sample the pressure at the cushion or cuff and the output of the piezoelectric transducers. The operation of the preferred embodiment is described as follows: Increasing and subsequent decreasing external pressure is applied as described for previous embodiments herein above. The pressure sensor 304 generates data similar to the curve shown in FIG. 4, which is used to find the desired test pressure and optionally to measure the subject blood pressure. As the artery recovers from collapse and the elementary blood volume progresses in the artery, consecutive piezoelectric transducers 306 are triggered and generate output signals, which are recorded by data acquisition system 308. The analysis of the data form the preferred embodiment shown in FIGS. 30a-b is described next.

1) The desired test pressure is found from the pressure data as described herein above.

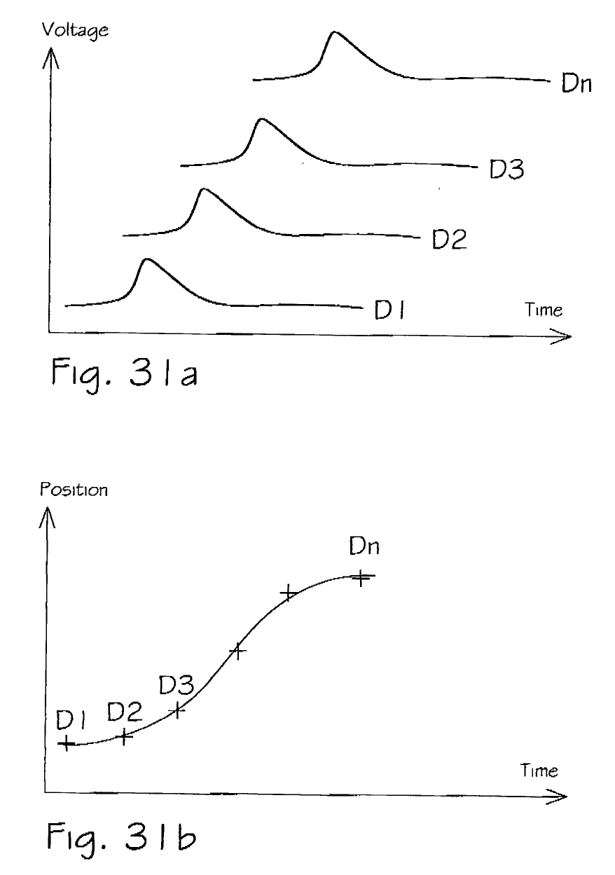

2) The acquired time-voltage curves (FIG. 31a) for each of the piezoelectric transducers are analyzed to determine the time of the pulse. One way to define the time is the crossing point of the leading edge of the pulse and a pre-set threshold. A more elaborated way is to determine the time at which the leading edge reaches a pre-set fraction of the pulse amplitude. This determined time corresponds to the time at which the blood entering the artery while opening the collapse reaches the specific transducer.

3) A graph of pulse time versus transducer position is generated where the position relative to the first transducer (most proximal) is known from the structure of the apparatus. See FIG. 31b.

4) The data is fitted to the empirical curve provided herein above for the pressure sensors data (FIG. 18). The Peripheral Velocity Time Integral PVTI is determined as the saturation value of the fitted curve.

5) The procedure may be repeated and results calculated for several pulses (typically 5) above and below the optimal test pressure. The PVTI value for the desired test pressure is determined by a polynomial fit of the PVTI values above and below the test pressure.

The preferred embodiments described herein above are designed to follow the method of first increasing the external pressure above the desired test pressure and than gradually decreasing it while acquiring data. The test pressure where the artery fully collapsed just momentarily in each heart cycle is found from the acquired data and results are computed. It will be appreciated that the desired test pressure can also be found during gradual increase of the external pressure provided the means for generating the pressure do not interfere with the measurement. For example, if the external pressure is generated by inflating a cuff, the pump should provide smooth monotonic increase of the pressure and be shielded electronically from the sensors readout electronics. The advantage of such arrangement is that measurements can be taken more frequently by periodic increase and decrease of the pressure.

Embodiments based on pressure increase and decrease are most suitable for applications whereby the apparatus is programmed to repeat the measurement periodically at pre-set time intervals, or to provide a single measurement per user request. The control unit may be provided with means for the operators to program the measurement frequency and to initiate a single measurement.

However, any of the preferred embodiments herein above can be operated also for continuous monitoring of the hemodynamic parameters of interest. To this end, the external pressure has to be kept at approximately the optimal test pressure and acquisition of the sensor data is continuous. Under such conditions, the Peripheral Velocity Time Integral (PVTI) can be computed for each pressure pulse and can be stored, processed and displayed as a function of time.

The following algorithm can be used for controlling the external pressure in embodiments for continuous monitoring of the blood flow:

1. The test pressure is first found by overshooting it during monotonic pressure increase by observing the first pulses for which there is a separation between the falling and rising edges of subsequent pulses (see FIGS. 14,15). The pressure is than reduced back to the desired test pressure.

2. The separation is calculated from pulse to pulse. If a separation is detected, the external pressure is decreased till there is no separation and than slightly increased to the desired test pressure.

3. Periodically, if there is no separation between pulses observed, the external pressure is increased till a separation is observed and than reduced back.

4. No hemodynamic data is calculated for the short time intervals while the pressure is adjusted.

The reader will appreciate that other procedures and algorithms can be applied as well to control the external pressure and are covered in the scope of the invention.

Any of the preferred embodiments inhere above can be provided with a memory unit to store the results of past measurement and later on display or transmit the patient past record. In particular, it is advantageous to store the results of the measurement for the patient while at rest in normal condition as a baseline to compare to further measurements during a condition of suspected decrease in cardiac output.

Furthermore, any of the preferred embodiments inhere above can be provided with means to transmit the results of a recent measurement or the stored history data via telephone line, cellular telephone system, cord or cord-less communication line to a computer, direct link to computer network or any other mean of electronic communication.

Furthermore, any of the preferred embodiments inhere above can be provided with means to generate visible or audible alarm in case it identifies measurement results which indicated a possible situation of myocardial ischemia. It is useful to store baseline normal condition data as a reference to detect abnormal results. The definition of alarming condition may be dependent not only on the PVTI but also on other parameters measured by the device such as heart rate and blood pressure.

Furthermore, any of the preferred embodiments inhere above can be integrated with other monitoring systems measuring other parameters to provide a complementary measurement. In some embodiments, the monitoring systems are ECG based monitors in hospital intensive care units. In other embodiments these are Holter systems used to monitor patients while they are carrying out their daily activity. The advantage of adding blood flow data to the ECG based monitoring is that false ECG alarms can be avoided by correlating the ECG with blood flow data.

It should be clear that the description of the embodiments and attached Figures set forth in this specification serves only for a better understanding of the invention, without limiting its scope.

It should also be clear that a person skilled in the art, after reading the present specification could make adjustments or amendments to the attached Figures and above described embodiments that would still be covered by the scope of the present invention.

1. A non-invasive apparatus for measuring cardiac mechanical performance of a patient, the apparatus comprising:
a pressure applying element mountable on a limb of the patient for applying pressure high enough to make a segment of an artery within the limb achieve a collapsed state and empty it from blood at least momentarily;
at least one of a plurality of sensors coupled to said pressure applying element, sensing mechanical changes corresponding to volumetric changes in the artery as the artery progressively recuperates from its collapsed state;
processing unit communicating with said at least one of a plurality of sensors for receiving output corresponding to the mechanical changes from said at least one of a plurality of sensors and computing factors correlated with blood flow and calculate parameters indicating heart performance.

2. The apparatus as claimed in claim 1, wherein the pressure applying element is an inflatable cuff.

3. The apparatus as claimed in claim 1, wherein the pressure applying element is an inflatable cuff, divided into a plurality of inflatable segments.

4. The apparatus as claimed in claim 3, wherein the inflatable cuff is divided into at least two inflatable segments, and wherein said at least one of a plurality of sensors comprise at least two sensor transducers for detecting pressure changes within the segment, each transducer corresponding to a different segment.

5. The apparatus as claimed in claim 3, wherein the pressure applying element is operated by a pneumatic system comprising a pump for increasing the pressure within the cuff, and valves for releasing the pressure from the cuff.

6. The apparatus as claimed in claim 1, wherein the pressure applying element is driven by an electrical motor.

7. The apparatus as claimed in claim 1, wherein the pressure applying element is coupled to a bracelet having a diameter which is automatically adjustable.

8. The apparatus as claimed in claim 7, wherein the bracelet consists of a strap and wherein bracelet's diameter may be increased or decreased by turning a screw operated by a motor to which the strap is attached.

9. The apparatus as claimed in claim 7, wherein the pressure applying element is hydraulically operated.

10. The apparatus as claimed in claim 1 wherein the pressure applying element comprises said at least one of the plurality of cushions held against the limb by a rigid bridge.

11. The apparatus as claimed in claim 10, wherein the cushions are inflatable.

12. The apparatus as claimed in claim 10, wherein said at least one of the plurality of cushions consist of two such cushions, filled with filled with ferromagnetic fluid that transforms from liquid to solid by application of magnetic flux, and electromagnetic coil provided adjacent each cushion, for inducing magnetic flux.

13. The apparatus as claimed in claim 1, wherein the pressure applying element comprises at least one of a plurality of cushions held against the limb by a rigid bridge, and wherein said at least one of a plurality of sensors comprises deformation sensors, sensing deformation changes of said at least one of the plurality of cushions.

14. The apparatus as claimed in claim 13, wherein said at least one of the plurality of cushions is inflatable.

15. The apparatus as claimed in claim 13, wherein said at least one of the plurality of cushions is filled with hydraulic fluid.

16. The apparatus as claimed in claim 13, wherein the deformation sensors comprise an array of capacitors, wherein the mechanical changes are determined by measuring changes in the capacitance of the capacitors, due to deformation changes.

17. The apparatus as claimed in claim 1 wherein said at least one of a plurality of sensors include an array of piezoelectric transducers wherein the mechanical changes are determined by measuring changes in the output voltage of the transducers.

18. The apparatus as claimed in claim 1, wherein the pressure applying element comprises at least one cushion held against the limb by at least one of a plurality of pivotal rigid bridges, each provided with gyroscopic sensor to sense rotational velocity of said at least one of a plurality of pivotal rigid bridges.

19. The apparatus as claimed in claim 18, wherein said at least one of a plurality of pivotal rigid bridges comprise two pivotal bridges.

20. The apparatus as claimed in claim 19, wherein the two pivotal bridges are coupled to a third pivotal bridge.

21. The apparatus as claimed in claim 1, further comprising output means.

22. The apparatus as claimed in claim 1, further comprising memory unit.

23. The apparatus as claimed in claim 1, further comprising means to communicate with a computer, network or a telephone system.

24. The apparatus as claimed in claim 1, wherein the pressure applying element is capable of applying pressure sufficient to cause a collapse of the artery just momentarily during a diastolic phase of the patient.

25. The apparatus as claimed in claim 1, wherein the processing unit includes algorithm comprising the following steps:
a. calculating instantaneous pressure changes within the pressure inducing member as a function of time;
b. dividing the instantaneous pressure changes into segments corresponding to pulse rate periods of the patient;
c. finding the highest pressure at which there exists no separation between the falling edge and leading edge of two consecutive segments of the normalized instantaneous pressure changes and analyzing at least one segment located within 5 pulse rates from the two consecutive segments.

26. The apparatus as claimed in claim 25, wherein the algorithm included in the processing means further comprises, in the presence of noise, measuring and tabulating values of time elapsed between two pulses at a predetermined threshold and extrapolating the highest pressure at which there exists no separation between the falling edge and leading edge of two consecutive segments of the normalized instantaneous pressure changes.

27. The apparatus as claimed in claim 25, wherein the highest pressure at which there exists no separation between the falling edge and leading edge of two consecutive segments of the normalized instantaneous pressure changes is found by first increasing the applied pressure above the desired pressure and than acquiring pressure data while gradually reducing the applied pressure.

28. The apparatus as claimed in claim 25, wherein the highest pressure at which there exists no separation between the falling edge and leading edge of two consecutive segments of the normalized instantaneous pressure changes is found by gradually increasing the applied pressure while acquiring pressure data.

29. The apparatus as claimed in claim 25, wherein a control system is used to maintain the applied pressure over a period of time substantially at the highest pressure at which where there exists no separation between the falling edge and leading edge of two consecutive segments of the normalized instantaneous pressure and factors correlated with blood flow are measured continuously.

30. The apparatus as claimed in claim 1, wherein the measurement data is used to calculate the peripheral velocity time integral PVTI.

31. The apparatus as claimed in claim 3 or 30, wherein the PVTI is calculated by a fit of a theoretical curve to the combined data of plurality of sensors, each detecting pressure changes within corresponding segment of the inflatable cuff.

32. The apparatus as claimed in claim 3 or 30, wherein the PVTI is calculated from the time difference between data of plurality of sensors, each detecting pressure changes within corresponding segment of the inflatable cuff.

33. The apparatus as claimed in claim 30, wherein the PVTI is calculated by a fit of a theoretical curve to data indicating sensor segment triggering time versus said segment position.

34. The apparatus as claimed in claim 30, wherein the PVTI data is used to calculate further factors correlated with blood flow.

35. A method for non-invasive measuring of changes in cardiac mechanical performance of a patient, the method comprising:
providing a pressure applying element mountable on a limb of the patient for applying pressure enough to make a longitudinal segment of an artery within the limb achieve a collapsed state and empty it from blood at least momentarily;
providing sensor coupled to the pressure applying element, sensing mechanical changes corresponding to volumetric changes in the artery as the artery progressively recuperates from its collapsed state;
providing processing unit communicating with the sensor for receiving output corresponding to the mechanical changes from the sensor and computing factors correlated with blood flow and calculate parameters indicating heart performance;
applying pressure on a portion a limb of a patient through which artery passes enough to collapse the artery preventing at least momentarily the flow of blood through the collapsed artery;
sensing mechanical changes corresponding to volumetric changes in the artery as the artery progressively recuperates from its collapsed state;
computing factors correlated with blood flow and calculating parameters indicating heart performance.

36. The method as claimed in claim 35, wherein the pressure applied on the portion of the limb of the patient is initially larger than needed to collapse the artery, and wherein it is gradually reduced, sensing the mechanical changes correlating to the volumetric changes while the pressure is reduced.

37. The method as claimed in claim 35, further comprising determining a best pulse period for considering a measurement, comprising the steps of:
a. calculating instantaneous pressure changes within the cuff as a function of time;
b. dividing the instantaneous pressure changes into segments corresponding to pulse rate periods of the patient and normalizing the pressure changes of each time segment;
c. finding two consecutive segments of the normalized instantaneous pressure changes where there exists no separation and analyzing at least one segment located within 5 pulse rates from the two consecutive segments.

38. The method as claimed in claim 35, further comprising measuring blood pressure of the patient.

39. The method as claimed in claim 35, further comprising measuring heart pulse rate of the patient.

40. The method as claimed in claim 35, carried out continuously over a period of time..

41. The method as claimed in claim 35, further comprising transmitting data to an external apparatus.

42. The method as claimed in claim 35, wherein it is incorporated with Holter procedure, in order to detect artifacts and enhance reliability.
